(19) United States
(12) Patent Application Publication
(10) Pub. No.: US 2024/0059846 A1
(43) Pub. Date: Feb. 22, 2024

(54) REACTIVE MICROPARTICLES AND THEIR USE TO PREPARE FUNCTIONAL HYDROGEL PARTICLES

(71) Applicant: ALLARTA LIFE SCIENCE INC., Dundas (CA)

(72) Inventors: Mitchell JOHNSON, Hamilton (CA); Samantha ROS, Hamilton (CA); Nicole MANGIACOTTE, Hamilton (CA); Nicholas BURKE, Dundas (CA); Harald STOVER, Dundas (CA)

(21) Appl. No.: 18/256,329

(22) PCT Filed: Dec. 7, 2021
(86) PCT No.: PCT/CA2021/051758

§ 371 Date: Jun. 7, 2023

Related U.S. Application Data
(60) Provisional application No. 63/122,322, filed on Dec. 7, 2020.

(51) Int. Cl. C08J 3/14; A01N 1/02; B01J 13/04; C08J 3/075; C08J 3/24; C12N 1/04; C12N 11/04

(52) U.S. Cl. CPC C08J 3/14; A01N 1/0221; B01J 13/046; C08J 3/075; C08J 3/243; C12N 1/04; C12N 11/04; A61K 2039/6093; C08J 2335/02

(57) ABSTRACT

There is provided a method for producing hydrogel microparticles with spherical shape and having a narrow-disperse or mono-disperse size distribution. At least one temporary crosslinker such as those of formula (I), (Ila)-(Ilf) and at least one permanent crosslinker comprising two or more vinyl groups, such as: divinylbenzene (DVB), ethylene glycol dimethacrylate (EGDMA),diethyleneglycol dimethacrylate (DEGDMA), N,N′-methylenebisacrylamide (MBA), oligo/poly ethyleneglycol dimethyacrylate, 1,4-butanediol dimethacrylate, and 1,6-hexanediol dimethacrylate are combined in an organic solvent having a polarity suitable for a precipitation polymerization to occur. The precipitation polymerization is allowed to take place without the addition of surfactant and/or stabilizer and/or the formed microparticles comprise less than 1% surfactant and/or stabilizer. These microparticles may be further functionalized to obtain amine and carboxylic acid units by functionalizing the monomers of the temporary crosslinkers. The functionalized microparticles are used for cryopreserving cells or as a vaccine delivery platform.

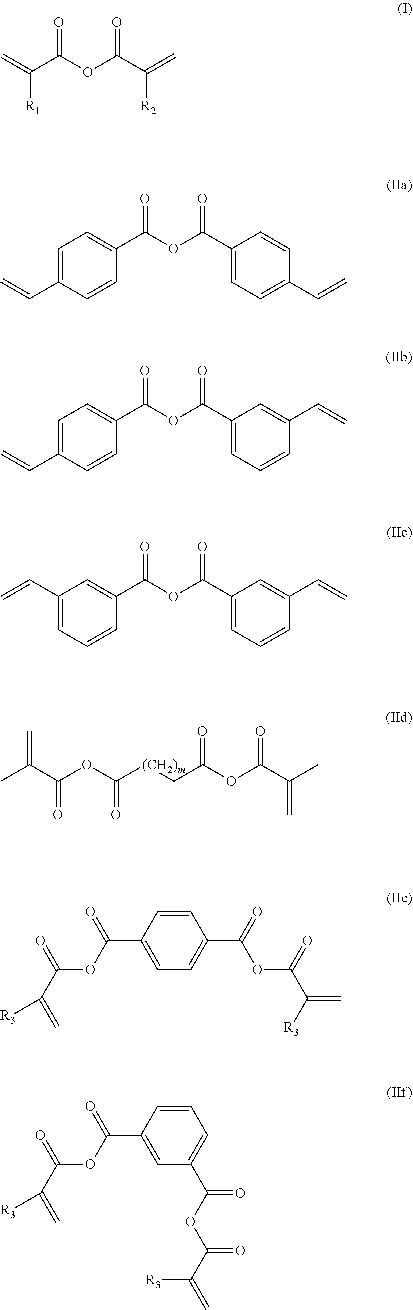

This application claims priority to U.S. provisional patent application 63/122,322 filed Dec. 7, 2020, the contents of which are incorporated herein by reference.

### FIELD OF THE INVENTION

The present invention is directed toward the formation and use of hydrogel microparticles bearing charged groups. More particularly, the invention relates to the formation of reactive, crosslinked microparticles that may be converted into crosslinked, functional hydrogel microparticles.

### DESCRIPTION OF THE RELATED ART

Hydrogels have long been recognized as useful materials to interface with cells and tissues as it is recognized that hydrogels can mimic certain properties of natural tissue, and can hence be thought of as synthetic extracellular matrix (ECM) materials. Examples range from synthetic crosslinked hydrogels based on hydroxyethyl methacrylate (HEMA) used as contact lenses, to Matrigel™, a commercially available ECM derived from mouse cancer cell-lines that allows culturing stem cells without inducing differentiation, and many forms of polyethylene glycol-based hydrogels.

In addition to bulk hydrogels, there have been many techniques and applications described for hydrogels formed as irregular or spherical particles, with narrow or broad size distribution, in the nanometer and micrometer size range. Such microgel particles have received great interest, due to their many potential applications.

Microgels have been used as cryoprotective materials for cryostorage of mammalian cells. For example, betaine-functional crosslinked hydrogels have been mechanically broken down into irregular microparticles that have shown cryoprotective properties. Cell attachment within matrices constructed of inverse suspension microgels with broad particle size distribution have been shown.¹

Some polymer particles in the micron and submicron range have found use as virus like particles (VLPs), enhancing antigen uptake by the host's immune system.

There is a need to be able to fine-tune the properties of hydrogels, both bulk and microparticle, such that they have the suitable mechanical properties and present chemical and biological groups appropriate to the biomedical application. One way of fine-tuning the hydrogel properties is through the use of a reactive precursor particle that can be functionalized as desired prior to hydrogel formation. Polymer particles are typically made by suspension, emulsion, dispersion or precipitation polymerization.

Polymer particles may be obtained by breaking a larger polymer solid or gel into small pieces, or by controlled phase separation of a preformed polymer from solution. However, most often, polymer particles with spherical shapes are formed using a particle-forming polymerization method such as suspension, inverse (water-in-oil) suspension, emulsion, inverse emulsion, dispersion or precipitation type polymerizations.

Suspension and inverse suspension polymer particles have homogeneous particle properties, as they are formed in essentially mini-bulk polymerizations; however, residual stabilizer on their surfaces can affect their interaction with cells and tissue. As well, suspension and inverse suspension polymerization carried out using mechanical dispersal of the liquid particle forming phase (e.g., monomer mixture) in a bulk continuous phase usually produce particles with broad size distributions, given the statistical balance of droplet sharing and coalescence found in these processes.

Inverse suspension polymerizations of droplets of aqueous solutions of hydrophilic monomers mechanically dispersed in an immiscible, oil-like media can be used to form spherical microparticles and microgels at large scale, though with broad size distributions.

Similarly, dispersal of aqueous coacervate phases in a continuous aqueous phase, followed by crosslinking of the dispersed droplets into hydrogel beads, can be seen as an example of aqueous-aqueous suspension polymerization leading to spherical, crosslinked hydrogel particles, though again with large particle size distributions.^{2,3}

Emulsion type polymerizations use particle initiation in the continuous media, and can result in the large-scale production of narrow-disperse nanoparticles. In both suspension and emulsion type polymerizations, water is typically used as the solvent, which may not be amenable to reactive, hydrolytically unstable monomers.

Dispersion polymerization starts with a solution of monomers, initiators and colloidal stabilizers in solvents that are poor for the forming polymer. This process takes advantage of the decreasing solubility of growing polymer chains, and can be used to form mono-disperse microparticles if large amounts of steric stabilizers are used to prevent aggregation of the forming particles.

Microfluidic particle formation is a version of suspension polymerization that involves one-by-one formation of micrometer-range droplets of monomer or polymer solutions suspended in a continuous medium, followed by rapid curing or crosslinking. Such methods have been used to prepare narrow and mono-disperse hydrogel particles for use as supports in culture of beta cells.⁴ The ability of 20 micrometer hydrogel beads was demonstrated to support cell attachment through RGD cell attachment motifs, and to increase cell viability which was attributed to a number of factors including better oxygen diffusion. Disadvantages of this approach are the limited through-put given the particle-at-a-time formation principle, the need for stabilizers, and the inability to produce particles with radial crosslink or other compositional gradients.

The stabilizers and surfactants used in dispersion, suspension, inverse suspension and emulsion polymerizations, including microfluidic variants, can be incorporated into the particles and their presence, in particular at the particle surface, can affect subsequent applications of the particles.

Precipitation polymerization is a variant of dispersion polymerization without added colloidal stabilizer. As a result, the forming polymer chains aggregate in an uncontrolled fashion, leading to irregular shaped particles with a broad size distribution.

Controlled precipitation polymerization is a variant of precipitation polymerization, typically using a significant crosslinker loading, where the polarity and hydrogen bonding abilities of the solvent or solvents are adjusted relative to those of the forming polymers such that the forming polymer chains assemble into nuclei that are colloidally stabilized by their solvated surface layer of just-absorbed chains. These particle nuclei subsequently grow in parallel by absorbing more polymer and monomer to form a final set of microparticles in the 0.3 to 20 micrometer diameter range.

Overall monomer loading in precipitation polymerization tends to be limited to 2-20, and more normally 2-10, weight percent total monomer in solvent. Higher monomer loading typically leads to formation of particle aggregates, while lower monomer loading leads to low yields due to inefficient particle nucleation and growth.

Those skilled in the art can adjust the solvent polarity to influence the number of polymer nuclei present at the point where this colloidal stabilization takes place, thereby controlling the final particle size. One of the monomers used in precipitation polymerization must be a crosslinker. Presence of such crosslinkers is critical to capture oligomers into nuclei, and subsequently onto the growing particles. Presence of crosslinkers also helps maintain particle integrity during growth.

Particle yield in precipitation polymerizations is highest at high crosslinker content relative to other monomers. However, particles made with high crosslinker loading are typically less deformable than desirable for targeted applications. When low crosslinker content is used the yield obtained is often quite low (e.g., <10%)⁵, and would only be suitable at small scale (i.e., experimental or laboratory). It would not be possible to obtain a commercially and economically viable method when the yield is too low due to the low crosslinker content.

Lightly crosslinked, swellable particles are hence formed typically in low yields by this method, as the low crosslinking density required for swellable particles conflicts with the need for large amounts of crosslinker to be present during polymerization to form particles in high yield. It is necessary to obtain a high yield in order to derive any usefulness at industrial scale.

Functional groups are generally introduced through selection of appropriate comonomers, rather than through post-functionalization of pre-formed particles. As such, means of functionalizing microgel particles to suit specific needs are not always available.

### SUMMARY

In one aspect, there is provided a method for producing microparticles comprising: combining at least one temporary crosslinker and at least one permanent crosslinker in an organic solvent having a polarity suitable for a controlled precipitation polymerization to occur; and allowing the precipitation polymerization to take place thereby forming the microparticles having polymers comprising monomers of the temporary crosslinkers and the permanent crosslinkers.

In one embodiment, a total monomer loading before the precipitation polymerization is calculated as the combined loading of the at least one temporary crosslinker, the at least one permanent crosslinker, and any other monomers, and has a value of between 1 to 20 weight %.

In one embodiment, a total crosslinker loading before the precipitation polymerization is the combined loading of temporary crosslinker and permanent crosslinker and has a value of more than 10 mol %, and wherein the ratio of temporary crosslinker to permanent cross linker is between 50:50 and 99:1 mol %.

In one embodiment, the solvent is 4 to 5 MPa^{1/2} above or below that of the polymers. In one embodiment, the solvent is selected from the group consisting of acetonitrile, methyl ethyl ketone, heptane, and combinations thereof. In a further embodiment, the solvent is selected from the group consisting of acetonitrile, methyl ethyl ketone, heptane, and combinations of methyl ethyl ketone and heptane.

In one embodiment, the temporary crosslinker is of formula (I) or (Ila)-(Ilf)

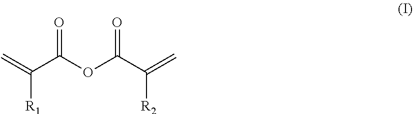

wherein R₁ and R₂ are independently selected from H, C₁-C₄ linear or branched carbon chain, benzyl, phenyl or OJ, where J is defined as a C₁-C₄ linear or branched carbon chain;

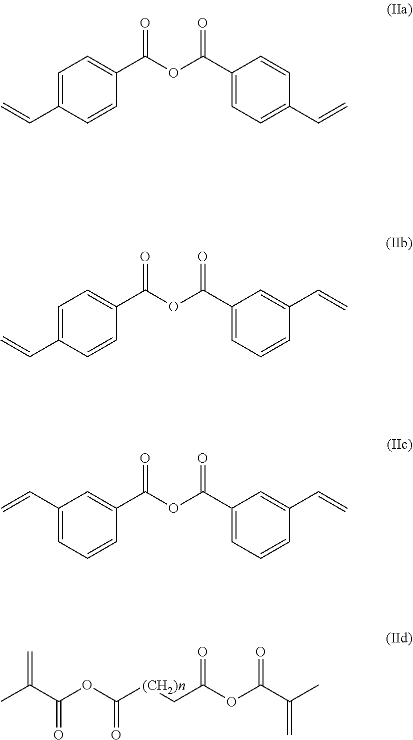

and wherein n is an integer from 1 to 3;

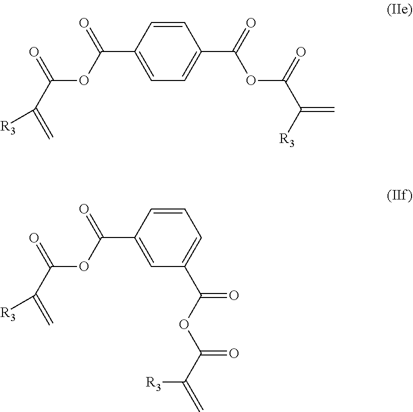

where R₃ is independently H or methyl.

In one embodiment, the temporary crosslinker is methacrylic anhydride or acrylic anhydride.

In one embodiment, the permanent crosslinker has two or more vinyl groups. In one embodiment, the permanent crosslinker is selected from the group consisting of divinylbenzene (DVB), ethylene glycol dimethacrylate (EGDMA), diethyleneglycol dimethacrylate (DEGDMA), and N,N′-methylenebisacrylamide (MBA).

In one embodiment, the permanent crosslinker is between 1 to 30 mol % of the total monomer loading.

In one embodiment, the yield of the microparticles is at least 30%, and preferably at least 50%.

In one embodiment, precipitation polymerization is performed in absence of surfactant and/or stabilizer. In one embodiment, the method is performed without the addition of surfactant and/or stabilizer.

In one embodiment, the microparticles have an outer surface comprising less than 3% surfactant and/or stabilizer.

In one embodiment, the method further comprises functionalizing the monomer units within the particle derived from the temporary crosslinkers.

In one embodiment, the step of functionalizing comprises functionalizing to obtain amines and carboxylic acid units in a ratio of 3:1 to 1:3. In one embodiment, the ratio is between 2:1 to 1:2.

In a further aspect, there is provided microparticles comprising at least one polymer, the at least one polymer comprising:
a temporary crosslinker monomer of formula (Illa), (Illb), (Illc), (Illd), (Ille), (Illf), (Illg), (Illh), (Illi), (Illj), (Illk), (Illl), (Illm), (Illn), and/or (Illo):

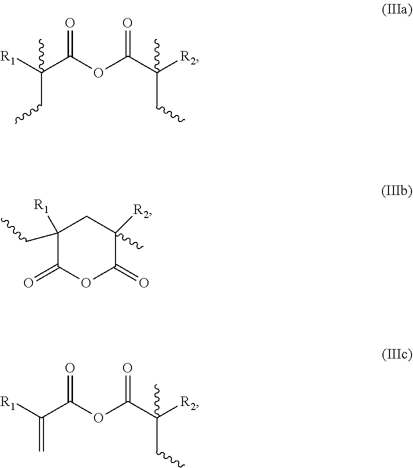

wherein R₁ and R₂ are independently selected from H, C₁-C₄ linear or branched carbon chain, benzyl, phenyl, or from —OJ, and wherein J is defined as a C₁-C₄ linear or branched carbon chain, with the wavy lines standing in for the rest of the polymer backbone,

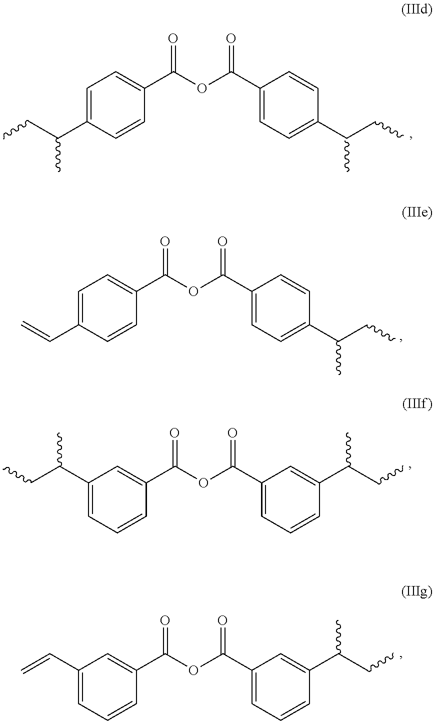

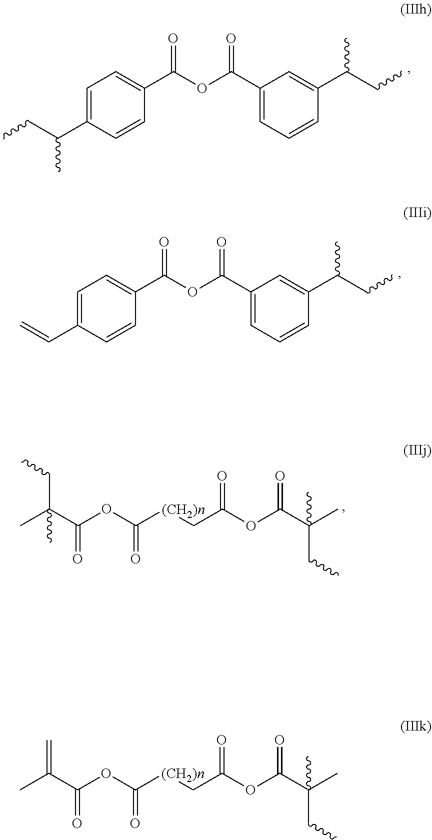

wherein n is an integer from 1 to 3.

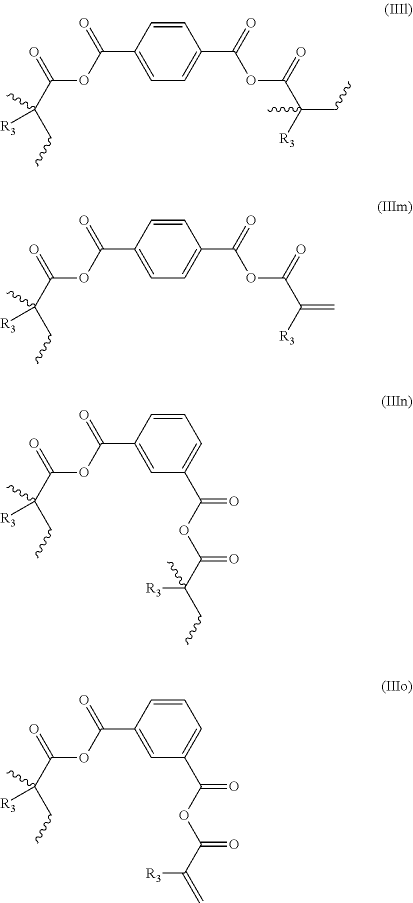

where R₃ is independently H or methyl, a permanent crosslinking monomer; wherein the microparticles are narrow-disperse or mono-disperse and have a size distribution having a coefficient of variation of less than 0.3.

In one embodiment, the hydrogel microparticles include no detectable surfactant or stabilizer.

In some embodiments, the hydrogel microparticles comprise less than 3%, preferably less than 1% of a surfactant and/or a stabilizer.

In some embodiments, the hydrogel microparticles have a surface and a core and less than 3%, preferably less than 1% of the surface area is a surfactant and/or a stabilizer.

In one embodiment, the hydrogel microparticles have a swelling ratio of wet to dry of between 5:1 to 50:1.

In one embodiment, the hydrogel microparticles have a total crosslinker content relative to a total monomer content of between 1 to 20 mol %, and preferably 5 to 15 mol %.

In one embodiment, the hydrogel microparticles have a deformability of between 1 kPa to 500 kPa, and preferably 10 to 100 kPa.

In one embodiment, the hydrogel microparticles have a spherical shape when swollen in aqueous media.

In one embodiment, the hydrogel microparticles have a diameter of between 0.5-20 micrometer.

In one embodiment, the hydrogel microparticles have a diameter of between 1 and 10 micrometer.

In one embodiment, the permanent crosslinker monomers are monomers of divinylbenzene (DVB), ethylene glycol dimethacrylate (EGDMA), diethyleneglycol dimethacrylate (DEGDMA), oligo/poly ethyleneglycol dimethyacrylate, 1,4-butanediol dimethacrylate, 1,6-hexanediol dimethacrylate, N,N′-methylenebisacrylamide (MBA), oligo/poly ethyleneglycol dimethyacrylate, 1,4-butanediol dimethacrylate, and 1,6-hexanediol dimethacrylate.

In one embodiment, the temporary crosslinker monomers are monomers of methacrylic anhydride and/or acrylic anhydride.

In yet a further aspect there is provided microparticles produced by the method described herein.

In an additional aspect, there is provided a method of cryopreserving cells comprising: providing microparticles as described herein; functionalizing the microparticles; contacting the cells with the microparticles; and freezing the cells.

In yet an additional aspect, there is provided a method of producing a vaccine delivery platform comprising: providing microparticles as described herein; functionalizing the microparticles to act as a carrier for an antigen; and associating the antigen to the carrier.

In a further aspect, there is provided a method of producing encapsulated cells comprising: providing the microparticles as described herein; functionalizing the microparticles; combining functionalized microparticles with cells and a capsule-forming material; gelling the capsule-forming material such that the particles and cells become entrapped within the capsule. In one embodiment, the capsule-forming material is alginate.

In yet a further aspect, there is provided a cryopreservative for cells comprising: a

monodisperse composition of biocompatible polyampholyte hydrogel microparticles, the hydrogel microparticles having a deformability of between 100 Pa to 100 kPa, and preferably 1 to 10 kPa; being substantially free of surfactant or stabilizer; and having a swelling ratio of wet to dry of between 5:1 to 50:1.

In an additional aspect, there is provided a method of cryopreserving cells comprising combining the monodisperse composition of biocompatible polyampholyte hydrogel microparticles as described herein with cells in an aqueous suspension in a microparticle to cell volume ratio of 5000:1 to 10:1, preferably 1000:1 to 100:1, and freezing the suspension of microparticles and cells.

In one embodiment, the freezing is done at a rate of 1 degree Celsius per minute down to minus 80 degree Celsius, followed optionally by transfer of the cryotube into storage containers held at liquid nitrogen boil-off temperature.

In another embodiment, the cell suspension is frozen rapidly by immersion into an environment held at minus 70 to minus 80 degree centigrade.

In yet another embodiment, the cell suspension is frozen by immersion into an environment held at the boil-off temperature of liquid nitrogen, which is minus 195.6 degree centigrade at one atmosphere pressure.

In another embodiment, the cells are stem cells. In a further embodiment the cells are primary cells.

In another embodiment, the hydrogel microparticles are in a concentration of 1-25 wt/v %.

In another embodiment, the cells are clusters of cells, also known as organoids, comprising between 10 and 5000 cells, and preferably between 100 and 2000 cells each.

In yet an additional aspect, there is provided a vaccine delivery vehicle comprising: a monodisperse composition of biocompatible hydrogel microparticles, the hydrogel microparticles being cationic or polyampholytes having an excess of cationic charge; being substantially free of added surfactant or stabilizer; having a swelling ratio of wet to dry of between 5:1 to 50:1; and having an average particle diameter between 0.1 and 10 microns.

In one embodiment, the microparticles are degradable in physiological conditions, over a time span of between 30 minutes and 10 days, and preferably between 2 and 48 hours

In one aspect, there is provided a method of making a vaccine comprising combining the vaccine delivery vehicle as described herein with an antigen.

In a further aspect, there is provided a granular extracellular matrix comprising: a monodisperse composition of biocompatible hydrogel microparticles, the hydrogel microparticles having a deformability of between 100 to 100 kPa, and preferably 1 to 10 kPa; a surface substantially free of surfactant or stabilizer; and a swelling ratio of wet to dry of between 5:1 to 50:1. In one embodiment, the microparticles are modified with a cellular adhesion molecule.

In yet a further aspect, there is provided a method comprising adding the monodisperse composition of biocompatible hydrogel microparticles as described herein to a suspension of mammalian cells in a ratio of cells to microparticles of between 1:100 and 1:1 in a gel former and gelling the suspension.

In another aspect, there is provided a cell culture method comprising providing the granular extracellular matrix as described herein and growing a cell culture on the granular extracellular matrix.

In yet another aspect, there is provided a biomimetic bead comprising a biocompatible hydrogel microparticle, the hydrogel microparticle having a deformability of between 100 to 100 kPa, and preferably 1 to 10 kPa; being substantially free of added surfactant or stabilizer; and having a swelling ratio of wet to dry of between 5:1 to 50:1; and a biomimetic functional group.

In still another aspect, there is provided a cell culture method comprising providing the biomimetic bead as described herein to a cell culture, and growing the cell culture.

### BRIEF DESCRIPTION OF THE DRAWINGS

FIG. 1 is a reaction scheme showing polymerization of methacrylic anhydride (MeAn) showing cyclopolymerization, where the two vinyl groups are consumed in sequential reactions, or a more conventional reaction where only one vinyl group reacts; if the second vinyl group reacts at a later time, a crosslink is formed.

FIG. 2 is a reaction scheme showing reaction of methacrylic anhydride-based polymer with nucleophiles (RXH, where X may be O, N, or S for example), which could take the form of hydrolysis or functionalization. Reaction of anhydride bridging two chains will lead to loss of crosslink.

FIG. 3 shows three approaches to forming micron-range microgel particles with polyampholyte properties. All three approaches start with a precipitation polymerization on a temporary divinyl crosslinker (e.g., methacrylic anhydride, MeAn) together with a permanent crosslinker (e.g., diethyleneglycol dimethacrylate (DEGDMA)).

FIG. 4 shows two approaches to forming nanoparticles for use as antigen carriers for vaccine applications. Both include an initial precipitation polymerization of a temporary crosslinker (e.g., methacrylic anhydride), together with a slowly erodible divinyl crosslinker (e.g., a disulfide-bridged dimethacrylate) to ensure the particles will ultimately be removed by renal clearance.

FIG. 5 shows the formation and functionalization of a reactive particle platform composed of temporary and permanent crosslinkers to produce microgels.

FIGS. 6A-C show light microscope images of MeAN/DEGDMA (90:10) particles made in 60:40 MEK/heptane: 6A—after formation (anhydrides intact) in DMF; 6B—hydrolyzed particles (anhydrides cleaved) in phosphate-buffered saline (PBS) at pH 2; and 6C—hydrolyzed particles in PBS at pH 7.4. Size bars: 20 μm.

FIG. 7 shows a brightfield optical microscope image of MeAN-only (MED-55/0/0) microspheres formed in a 55/45 MEK/heptane in the absence of permanent crosslinkers. The particles were suspended in MEK for imaging.

FIGS. 8A-F shows brightfield optical microscope images of MED-55/5/5 particles made with 2 to 7% monomer loading in acetonitrile 100× oil immersion (8A: 2%, 8B: 3%, 8C: 4%, 8D: 5%, 8E: 6%, and 8F: 7%). Particle dispersed in ACN for imaging. Size bar: 10 μm.

FIG. 9 shows a graph of the diameter in function of the weight % of AlBN (initiator) demonstrating the effect of initiator loading on the size of MED-62/0/10 particles made by photopolymerization.

FIG. 10 shows a graph of the diameter as a function of the MEK vol. % in the solvent, demonstrating the effect of varying MEK/Heptane ratio on the size of MED-X/0/10 particles made by photopolymerization. Particles are formed with>62% MEK but they became gradually smaller and their size could not be accurately determined by optical microscopy (data points marked with “?”).

FIG. 11 shows brightfield optical microscope images of anionic MED-55 microgels made with different ratios of EGDMA/DEGDMA permanent crosslinker (10 mol % total). Top row—MED-55/10/0; Middle row—MED-55/5/5; Bottom row—MED-55/0/10. Scale bar=5 μm; Images cropped to magnify images.

FIG. 12 shows a graph of the swelling ratio in function of the pH for (MED-55/10/0 (▴) and MED-55/5/5 (▪)), demonstrating the effect of crosslinker composition on the swelling of anionic microgels as a function of pH. The swelling ratio is normalized to the particle volume at pH 2.4 using (Dₓ/D_{2.4})³, where Dₓ is the particle diameter at a given pH and D_{2.4} is the diameter at pH 2.4.

FIG. 13A shows an example of microgel particles formed by precipitation copolymerization of methacrylic anhydride (90 mol %) with DEGDMA (10 mol %) at 5 wt % total monomer loading in a 60:40 MEK:heptane mixture followed by functionalization with N,N-di methylethylenediamine to produce polyampholyte hydrogel particles. The particles are suspended in HEPES-buffered saline (pH 7.6).

FIG. 13B shows a graph of the distribution of particle sizes expressed as the particle area in μm²-. The majority of the particles have areas between 4 and 6 μm² corresponding to particle diameters of 2.25 to 2.75 μm.

FIG. 14A shows a brightfield microscopy image of MED-55/5/5 particles in DMF before hydrolysis. FIG. 14B shows a microscopy image of close-packed multilayer of DMAPA-and TAMRA-functionalized MED-55/5/5 particles in water. FIG. 14C shows a confocal fluorescence microscopy image of DMAPA- and TAMRA-functionalized MED-55/5/5 particles in water. Size bars: 15 μm.

FIG. 15 shows a bar graph of the zeta potential of MED-55/10/0 microspheres after hydrolysis and functionalization with DMAPA, measured in PBS at pH 7.4.

FIG. 16 is a ¹H-NMR (600 MHz) spectrum of (propane-2,2-diylbis(oxy))bis(ethane-2,1-diyl) bis(2-methylacrylate) (KTMA) in CDCl₃.

FIG. 17 shows an optical microscopy image of MKT-55/15 particles made with 85:15 MeAn/KTMA in 55:45 MEK/heptane. Size bar: 5 μm.

FIG. 18 shows brightfield optical microscope images of MKT-55/15 Anionic microgels over time at pH 5 (top row), pH 7 (middle row), and pH 10 (bottom row). Scale bar=15 μm.

FIG. 19 shows a graph of the immediate post-thaw cell viability and percentage of recovered 3T3 cells after a 24-hour freeze/thaw cycle for cells in the presence of: MED-55/2/8 10 wt % (▪), MED-55/2/8 5 wt % (♦), negative control (●), and DMSO 10 v/v % (▴).

FIG. 20 shows a graph of 3T3 cell numbers as a function of day post thawing for cells frozen with DMEM containing either, polyampholyte microgels or DMSO, and a negative control of cells frozen in DMEM without supplemental cryoprotective agents: MED-55/2/8 10 wt % (▪), MED-55/2/8 5 wt % (♦), negative control (●), and DMSO 10 v/v % (▴).

FIG. 21 shows brightfield microscopy images of thawed 3T3 cells after freezing with cryoprotective polyampholyte microgels.

FIGS. 22A-22B shows bright-field and fluorescence images of pDMAEA-grafted particles after exposure to fluorescein-labelled ovalbumin (OVA-FITC) in PBS at pH 7.4. 22A: pDMAEA-grafted particles—OVA-FITC bright-field image in PBS, pH 7.33 and 22B: pDMAEA-grafted particles—OVA-FITC fluorescence image in PBS, pH 7.40.

FIGS. 23A-C show confocal fluorescence images of fluorescently stained 3T3 cells co-cultured with TAMRA-labeled MED-55/5/5 microgels. 23A—DMAPA polyampholytes, 23B—RGD anionic, 23C—anionic.

FIG. 24 shows a confocal image of NIH 3T3 cells stained with Calcein-AM mixed with TAMRA labelled polyampholyte MED-55/15/0 microgels.

FIGS. 25A and 25B show confocal images of NIH 3T3 cells co-encapsulated in PLL/PM50 calcium alginate capsules with MED-55/10/0 polyampholyte microgels at a cell concentration of 2.0×10⁶ cells/mL and 0.5 wt/v % microgels and stained with Calcein-AM and Ethidium-homodimer LIVE/DEAD staining (25A: 100 μm scale bar, and 25B: 15 μm scale bar).

### DETAILED DESCRIPTION

The present invention involves several aspects.

In one aspect, there is provided new compositions of monomers and crosslinkers for precipitation polymerization, designed to enable formation of a new type of polymer microgel particle in high yield and through a scalable process, and that combine properties not previously accessible. These properties include, but are not limited to, polymer microparticles being lightly crosslinked, swellable, narrow- or mono-disperse, stabilizer-free, reactive, and optionally, degradable. Such particles can serve as a platform for highly defined hydrogel particles for use in different areas of biomedicine.

In other aspects, there is provided novel applications for nano and microgel particles, including as cryoprotective particles, granular ECM components, and charge-shifting vaccine platforms.

In some embodiments, the microgel particles are formed by new methods for precipitation polymerization taught herein.

The term “microgel” as used herein refers to lightly crosslinked polymer systems in the form of microparticles that are swollen by a solvent. The term “hydrogel” as used herein refers to lightly crosslinked polymer systems that are swollen in water.

The term “microparticle”, while generally used to refer to particles between 1 and 1000 μm in size, as used herein can also encompass, unless the context dictates otherwise, submicron particles in the 0.1 to 1 μm size (i.e., nanoparticles within this size range.) In preferred embodiments, the microparticles have a particle diameter between 0.1 and 50 μm, more preferably between 0.3 and 30 μm, or still more preferably between 0.5 and 20 μm.

The term “covalently crosslinked” as used herein with respect to a polymer matrix refers to the formation of covalent bonds between polymer chains that hold together the polymer matrix, a microparticle in this work. It is not possible for the polymer to undergo facile dissolution into individual polymer chains when the covalent crosslinks are present. In the case of the initially-formed reactive particles, the covalent crosslinks are provided by both temporary and permanent crosslinkers. After the temporary crosslinks have been cleaved by hydrolysis or functionalization, the overall network structure of the hydrogel particles is maintained by the covalent crosslinks provided by the permanent crosslinker.

In some variants, the permanent crosslinker is a slowly degradable crosslinker that can undergo degradation under physiological conditions, with timeframes on the order of 2 hours to 2 weeks, and preferably between 8 hour and 48 hours. Such slowly degradable crosslinker may be based on bisacrylate or bismethacrylate crosslinkers that contain a disulfide linkage, which may be cleaved over time under physiological conditions such as after administration into tissue during a vaccination, by reductive processes involving reaction with physiological glutathione that are chemically orthogonal to the processes used to chemically modify or hydrolyze the anhydride-based temporary crosslinkers. Other variants are described below, and include other bisacrylate or bismethacrylate crosslinkers containing other cleavable linkers between the two acrylate or methacrylate units. The purpose of using such slowly degrading crosslinkers is that they permit clearance of the particles after use.

The term “polyampholyte” as used herein refers to zwitterionic polymers which comprise monomer units with a positive charge and monomer units with a negative charge, wherein the positive and negative charges occur on different monomer units. The polyampholytes as discussed here are meant to be the copolymer comprising anionic and cationic comonomers (and optionally neutral and hydrophobic comonomers) that are grafted through the residual or introduced vinyl groups bound to the microgel particles. In one embodiment, polyampholytes comprise cationic units which are primary amines as well as anionic units which are carboxylic acids. Generally, the polyampholyte may comprise about 10-90 mol % of a positively charged monomer and 90-10 mol % of a negatively charged monomer, and preferably about 30-70 mol % of a positively charged monomer and about 70-30 mol % of a negatively charged monomer.

The final particles may contain at least 10% of polyampholyte by dry weight, preferably 50 to 400%.

In other embodiments, the cationic group can be a monomer comprising a secondary, tertiary or quaternary ammonium group, or a monomer comprising a guanidinium group, or a monomer comprising a sulfonium group, or a monomer comprising a conjugated diazole group such as found in imidazoles and analogous cyclic and linear groups known to those skilled in the art.

In other embodiments, the anionic group may be comprised of a monomer containing a carboxylic acid group such as acrylic acid, methacrylic acid, or precursors to such monomers such as t-butyl acrylate or t-butyl methacrylate.

In yet other embodiments, the hydrolyzed microgel acts as the polyanionic component, and the grafted polymer or copolymer acts as the cationic component. In such embodiments, the cationic component may be a homopolymer comprising cationic monomers incorporating a primary, secondary, tertiary or quaternary cationic monomer based on acrylate, methacrylate, acrylamide or methacrylamide polymerizable units. In related embodiments, the cationic component may be a copolymer comprising one or more of the above cationic monomers, together with a neutral or anionic comonomer. In such copolymers, the cationic monomer or monomers comprise 50 to 99, and preferably 70 to 90 mol % cationic comonomer.

In yet other embodiments, the polyampholyte microgel is formed by precipitation copolymerization of the temporary crosslinker with a cationic monomer and a permanent crosslinker, or a temporary crosslinker, a cationic monomer and a slowly degradable disulfide-containing crosslinker. In these embodiments the amounts of temporary crosslinker and cationic monomer are chosen such that the final anionic/cationic ratio can be controlled between 10/90 anionic/cationic and 50/50 anionic/cationic. Suitable cationic monomers for this embodiment include tertiary amines such as 2-(dimethylamino)ethyl methacrylate and N-(3-(dimethylamino)propyl)methacrylamide.

In yet other embodiments, the cationic polymer or copolymer is not grafted-through using residual or newly introduced vinyl groups, but rather is introduced by electrostatic complexation between the anionic hydrolyzed microgel particles, and the soluble cationic polymer or copolymers. As known by people skilled in the art, polyanionic polymers have a strong affinity to bind polycationic polymers to form polyelectrolyte complexes. This complexation between cationic or net cationic polymers with anionic polymer networks is driven by the associated release of small counterions from both participating charged polymers. Furthermore, it is known in the art that the resulting polyelectrolyte complexes may have physical properties spanning from solid precipitates of insoluble PECs, to liquid complex phases called complex coacervates, depending on the net strength of the electrostatic interaction between the two charged polymers. As further known in the art, polyanionic hydrogel particles have the ability to not only bind polycations comprised of only cationic monomers (cationic homopolymers), but also cationic copolymers that comprise mixtures of cationic monomers with hydrophilic neutral or even anionic comonomers. These copolymers would be non-stoichiometric polyampholytes defined as copolymers having an excess of cationic over anionic monomers in order to enhance absorption of the polyampholyte into the anionic microgel. Such non-stoichiometric polyampholytes may contain 30 to 99% cationic monomer, and preferably 50-80% cationic comonomer, and most preferably 60-70% cationic comonomer. They may also contain neutral and even hydrophobic comonomers, in addition to cationic and anionic monomers. Copolymers of a cationic monomer (3-aminopropylmethacrylamide, APM) with anionic monomer methacrylic acid⁶ or with a neutral hydrophilic comonomer N-(2-hydroxypropyl)methacrylamide (HPM)⁷ can be absorbed into calcium alginate hydrogel beads.

In some embodiments, the term “temporary crosslinker” as used herein refers to a crosslinker used to create a polymer particle that is yet to be functionalized. The temporary crosslinker is used to graft one or more functional groups such as amine, carboxyl, or thiol depending on the desired application for the polymer. The temporary crosslinkers can have an anhydride group and may be of formula (I) described below. In one embodiment, the temporary crosslinker is completely cleaved. However, even when the temporary crosslinker is completely cleaved the integrity of the polymer in solvent can be maintained thanks to the permanent crosslinker.

The term “permanent crosslinker” as defined herein refers to a crosslinker that will survive, largely intact, the conditions used to hydrolyze or functionalize a temporary crosslinker that is part of the same polymeric microparticle. For example, the conditions can be those to hydrolyze or functionalize anhydride groups. It may be one that itself can be cleaved under different conditions, or simply more slowly, as described below. In one embodiment, the permanent crosslinker has at least two vinyl groups. Examples include combinations of hydrolytically labile methacrylic anhydride, with hydrolytically stable mono-, di- and higher ethylene glycol dimethacrylates. In some embodiments, the permanent crosslinker is a degradable crosslinker or a biodegradable crosslinker. The degradable or biodegradable crosslinker does not react during the hydrolysis or the functionalization reaction of the temporary crosslinker but will degrade in vivo under physiological conditions. Such degradable crosslinkers include ketal or disulfide-containing crosslinkers that persist during hydrolytic cleavage of the temporary crosslinker but will degrade under physiological conditions over the course of hours to weeks.

The term “functionalize” or “functionalization” as used herein refers to a reaction where a functional group is produced from a reactive group. The functional group can be a peptide group (for example RGD) or other molecules (e.g., fluorophores, polymers, etc.). Functionalization includes reactions with nucleophiles like water (hydrolysis), amines, alcohols and thiols, which cleaves the anhydride group of the temporary crosslinker. In one example, thiols bearing hydrophobic, hydrophilic or biologically active groups can be used. Furthermore, the reaction may be with difunctional species, such as a diamine. When a difunctional species is involved in the functionalization reaction, defined for example as a diamine where both amines are primary or secondary amines such as 1,2-ethylenediamine or 1,3 propylenediamine, a new crosslink may be formed where the anhydride crosslinks would be broken and might be replaced with a diamide crosslink if both ends of the diamine reacted with anhydride groups. As well, the functionalization could involve diamines or tri amines or higher amines, wherein only one of the amine groups is a primary or secondary amine, and the other amine groups are tertiary or quaternary amine (ammonium) groups. An example would be N,N-dimethylamino propyl amine, and analogous di and higher amines known to people skilled in the art. A particular aspect of this functionalization with higher amines is the ability to introduce an excess of cationic over anionic groups into the hydrogel particle.

The term “biocompatible” as defined herein refers to compounds or microparticles that are compatible with in vitro or in vivo prolonged contact with cells and/or specific biological tissues. Biocompatible compounds or microparticles do not elicit a significant negative effect on the cell survivability, cell function, and/or tissue function, whereby biocompatibility is usually specified in terms of being compatible with a particular tissue or cell environment.

In one aspect, the present invention provides swellable, stabilizer-free, reactive, narrow size-disperse, nano- and microparticles in high yield, that can be modified to serve as useful agents for various biomedical applications.

This includes highly swellable microgel particles functionalized with amines and carboxylic acid units in an 80:20 to 20:80, and preferably in a 70:30 to 30:70 ratio.

These microgel particles may also be functionalized by grafting-through or grafting-from, using mixtures of anionic and cationic comonomers, again achieving an anionic to cationic charge ratio of about 80:20 to 20:80, and preferably 70:30 to 30:70.

Neutral hydrophilic monomers for such functionalizations include 2-hydroxyethyl methacrylate, 2-hydroxyethyl acrylate, acrylamide, methacrylamide, N,N-dimethylacrylamide, N,N-diethylacrylamide, N-isopropylacrylamide, (and other acrylamides/methacrylamides), PEG methacrylate, N-vinylpyrrolidone, and similar monomers known to people skilled in the art.

Neutral hydrophobic monomers include alkyl (C1-C12) methacrylates and acrylates, alkyl (C4-C12) methacrylamides and acrylamides, styrene, 4-methylstyrene, and other substituted styrenes.

Anionic monomers include acrylic acid, methacrylic acid, 2-carboxyethyl acrylate, 2-acrylamido-2-methylpropanesulfonic acid (or sodium salt), vinylsulfonic acid, styrenesulfonic acid (or sodium salts), vinyl-functional phosphoric and phosphonic acids such as, but not limited to, vinylphosphonic acid and 2-(methacryloyloxy)ethyl phosphoric acid.

Cationic monomers include N,N-dimethylaminoethyl methacrylate, N,N-dimethylaminoethyl acrylate, 3-(N,N-dimethylamino)propylmethacrylamide, 3-aminopropylmethacrylamide, 2-(methacryloyloxyethyl)trimethylammonium chloride, 3-(methacrylamidopropyl)trimethylammonium chloride (all represented in the general structures shown below), and vinylpyridine.

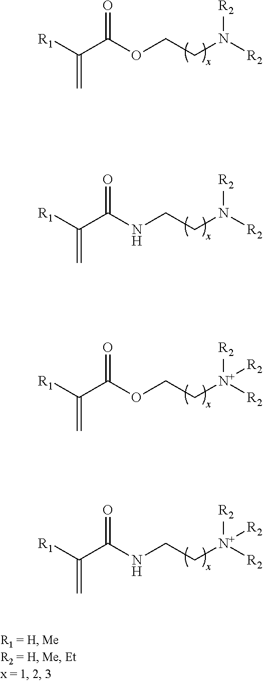

Zwitterionic monomers include 2-methacryloyloxyethyl phosphorylcholine, N-(2-methacryloyloxy)ethyl-N,N-dimethylammonio propanesulfonate, N-(3-methacryloylimino)propyl-N,N-dimethylammonio propanesulfonate, 3-(2′-vinyl-pyridinio)propanesulfonate, and 3-[[2-(methacryloyloxy)ethyl]-dimethylammonio]propionate (CBMA).

In some embodiments, the polymer may be grafted with monomers after functionalization. The grafted polymer network can for example contain hydrophobic monomers such as butyl acrylate, in amounts up to 50 mol %, and preferably up to 20 mol %. In some embodiments, the polymer network contains neutral hydrophilic comonomers designed to enhance the desired properties. Examples include addition of monomers bearing carbohydrate groups to enhance cryoprotective properties. Examples of neutral hydrophilic monomers include 2-hydroxyethyl methacrylate, 2-hydroxyethyl acrylate, acrylamide, methacrylamide, N,N-dimethylacrylamide, N,N-diethylacrylamide, N-isopropylacrylamide, (and other acrylamides/methacrylamides), and PEG methacrylate. Examples of neutral hydrophobic monomers include alkyl (C1-C12) methacrylates and acrylates, alkyl (C4-C12) methacrylamides and acrylamides, styrene, and 4-methylstyrene.

In prior art particle forming processes, it is necessary to add a stabilizer or surfactant (e.g., poly(vinyl alcohol), poly(vinylpyrrolidone), cellulose, sodium dodecylsulfate, etc.) to the polymerization mixture to successfully form particles. A portion of the added stabilizers or surfactants according to known methods are permanently bound to the particle or very difficult to remove. In the context of the stabilizers and surfactants, the term “add” or “added” as used herein refers to voluntarily adding stabilizers and/or surfactants to promote colloidal stability in the formation of microparticles. Microparticles precipitated according to methods taught herein without added stabilizers and/or surfactants are described as being “substantially free” of stabilizer or surfactant, which allows for the presence of a minimal amount of stabilizers (e.g., as an impurity). In the present methods of precipitation polymerization, no stabilizer or surfactant is added at any point in the process. Therefore, the particles are free of added stabilizers and/or surfactants. In some embodiments, surfactants and/or stabilizers are less than 0.1 wt. % of the total monomer. The surfaces of such precipitation polymerization particles are thus only defined by the comonomers and crosslinkers present during the particle formation, any initiator groups used to initiate the particle forming polymerization of the grafting-through polymerization, and any subsequent functionalization. In preferred embodiments, any stabilizer or surfactant is an impurity i.e. it is inadvertently added. In one embodiment, the particles according to the present disclosure comprise less than 1% of (an inadvertently added) surfactant or stabilizer. A person skilled in the art can determine the composition of a particle according to known methods in the art such as ¹H nuclear magnetic resonance spectroscopy or other chemical techniques such as coagulation.

The microparticles of the present invention are narrow disperse or monodisperse. In one embodiment, the microparticles have a size distribution having a coefficient of variation of about less than 0.3, about less than 0.2, or about less than 0.1.

The microparticles can be produced in a variety of different sizes while maintaining the advantageous narrow dispersity or mono dispersity.

By varying synthesis conditions, such as the nature of the solvent, the average particle size may be varied while maintaining a narrow-disperse size distribution (such as a coefficient of variation of less than about 0.3). The average diameter would be in the 0.2-20 um range, depending on solvent compositions. Solvent compositions can be adjusted to give narrow-disperse particles with diameters across this range. Within this overall range, particles with diameters ranging from 1 to 10 micrometers are most easily accessible.

The present microparticles can be characterized as swellable once the temporary crosslinks have been cleaved by hydrolysis or functionalization. In one embodiment, the microparticles have a swelling ratio of wet to dry of between about 3:1 to about 50:1. For some applications, including cryoprotection and cell chaperones, a swelling ratio of wet to dry is preferably 5:1 to 30:1. For applications such as vaccine encapsulation, a ratio of wet to dry of 3:1 to 10:1 is preferred. Thus, the swelling capacity of the microparticles can be adjusted for the specific application desired. The swelling of the particles contributes to the colloidal stability of the particles by limiting the phenomena of particles sticking to each other.

For biomaterials applications, lightly crosslinked, swellable hydrogels comprising 0.1-30 mol %, and preferably 2-10 mol %, permanent crosslinker relative to total monomer, are often desirable in order to better mimic tissue properties.

The present microparticles can be characterized as soft and lightly crosslinked. In one embodiment, a polymer of the microparticles comprises between 0.1 to 20 mol %, and preferably 1-10 mol %, of permanent crosslinkers relative to total monomer of the polymer. Functionally, “soft” can be defined as being biological tissue-like in terms of deformability. Quantitatively, “soft” can be defined as a deformability that is of the same order of magnitude as that of cells and tissues which is between about 100 Pascal (Pa) to about 100 kPa, about 5 to about 50 kPa, or about 1 to about 10 kPa.

The particles of the present disclosure generally have a spherical shape with a smooth or rough surface. In one embodiment, the shape is a sphere or an irregular sphere. The irregular sphere may be defined as having small bumps on the surface thereby rendering the surface rough. Without wishing to be bound by theory, the spherical nature or irregular sphere shape is explained by the way particles are formed with precipitation polymerization where the particles are grown by addition and are therefore driven towards a spherical shape. The shape may be advantageous in that it promotes close-packed arrays, both with other particles, and with cells.

Due to the properties detailed herein, the present microparticles and methods of producing can have many advantages:
The precipitation polymerization method described herein enables control of radial composition profiles including compositional and crosslink density profiles of microgel particles, which in turn enables better management of microgel-cell interactions.
The narrow size distribution may limit the deformation of admixed cells in comparison to irregular microparticles. Without wishing to be bound by a theory, the narrow size distribution allows the formation of close-packed arrays of particles, which provides consistent interstitial volumes between microgels. Irregular particles pack together with a range of interstitial volumes, some quite small, which in turn may increase the deformation of the admixed cells.
The surfactants or stabilizers typically present on particles formed by other polymerization techniques may affect and potentially dominate cell-particle interactions. In contrast, the absence of any added surfactant or stabilizer on the microparticle surface, a distinctive feature of precipitation polymerization, means that the interaction of cells with the microparticles are predominantly driven by the chemical and biological groups present on the particle surface as a result of the choice of monomers, initiators and functionalization reagents during the particle preparation.
The precipitation polymerization method described herein allows for a high through-put and high yield production of the microparticles.
These microparticles can be efficiently post-modified to tune particle properties. Examples of functionalizing agents include molecules containing a nucleophilic group comprising (primary or secondary) amine, hydroxyl, or thiol such as shown below

A—NH₂ A—OH A—SH

Where A=H, alkyl (linear and branched, C₁-C₁₂), phenyl, benzyl, dialkylaminoalkyl- or trialkylammonioalkyl, alkoxyethyl, oligo(ethyleneglycol).
For purposes of cell attachment, A may also be cell binding motifs such as RGD aminoacid sequences, as well as larger extracellular matrix components such as laminins. For purposes of study and particle tracking, A may also be a fluorescent group such as fluorescein or rhodamine, or other groups known to those skilled in the art.
Finally, these microparticles can be designed to change their charge balance, for instance by hydrolytic charge-shifting of the cationic components introduced during precipitation polymerization, post-functionalization, grafting-through as well as absorption of polycations. Examples include embodiments where the cationic components include charge-shifting cationic monomers such as dimethylaminoethyl acrylate (DMAEA) or other monomers and functional groups that are known in the art to undergo spontaneous hydrolysis of their ester linkage under physiological conditions with half-lives on the order of hours and days. Such groups may also be introduced during post-functionalization with, e.g., the lithium salt of N,N-dimethylaminoethanol into particles swollen in, e.g., tetrahydrofuran or 1,4-dioxane or similar solvents or solvent mixtures that are known in the art to be aprotic polar solvents. Such groups may also be introduced by grafting-through or grafting-from the particles with charge-shifting monomers such as dimethylaminoethyl acrylate, either by itself or in combination with other cationic, neutral, anionic or hydrophobic monomers designed to achieve a desired overall charge balance in the final particles. Such groups may also be introduced by electrostatic absorption of polymers comprising dimethylaminoethyl acrylate, either by itself or in combination with other cationic, neutral, anionic and/or hydrophobic monomers.

The final charge balance of the microgel particles produced by such functionalization, grafting or absorption of charge-shifting groups may comprise a majority of cationic charges for microgel designed for use in antigen binding for vaccine development, or have a near-stoichiometric ratio of cationic to anionic charges for microgel particles designed for cell cryoprotection.

### Precipitation Polymerization

There is growing interest in the use of mono-disperse, swellable, stabilizer-free hydrogel particles in biomedical applications, and, hence, there is a need for high yield methods to prepare such particles. Provided herein are high yield methods for producing mono-disperse, swellable, stabilizer-free hydrogel particles suitable for use in biomedical applications, that, advantageously, allow for the formation of hydrogel particles with a range of properties (e.g., size, stiffness, composition e.g., chemical/biological moieties present, radial distribution of properties such as crosslink density and composition). In one embodiment, the yield is defined as the weight or molar ratio of starting monomers and optionally initiators to monomers present in the polymer formed. In another embodiment, the yield is defined as the weight or molar ratio of starting monomers and optionally initiators to monomers present in the particles. In various embodiments, the yield can be at least at least 30%, at least 40%, at least 50%, at least 60%, and preferably at least 70% or at least 80%.

The hydrogel particles having the above-mentioned properties can be advantageously obtained by precipitation polymerization of one or more reactive monomer(s), under particle-forming conditions followed by hydrolysis and/or functionalization of that reactive monomer with suitable modifiers, and swelling in water.

Precipitation polymerization is well suited to making particles containing reactive monomer(s), in particular water-sensitive ones, as well as producing narrow-disperse, micron-sized particles that are free of stabilizers or surfactants. However, the relatively high levels of crosslinker required for efficient particle formation in precipitation polymerization would tend to give particles that were much stiffer than suitable for most biomaterial applications.

Precipitation polymerization begins with a homogeneous solution of monomers, at least one of which is a crosslinker, and an initiator. As polymer is formed, it precipitates from the solution. As evidenced in the Examples, under conditions provided herein, particles of the present disclosure are formed. These conditions typically include a total monomer loading between about 1 to about 20 wt %, or between about 2 to about 10 wt %, a crosslinker fraction (cf. total monomers) of between about 10 to about 100 mol % or between about 20 to about 80 mol % and, most importantly, a solvent with the right solvency properties for the polymer that is formed. The formation of particles becomes inefficient and limited with a total monomer loading lower than 1 wt %. In one embodiment, only crosslinkers are used in the loading. In one embodiment, one or more additional monomers (not a temporary crosslinker or a permanent crosslinker) may be added to the monomer loading to produce a polymer geared towards a specific application. In one embodiment, the ratio between temporary crosslinker to permanent crosslinker is between about 90:10 to about 80:20. The solvent should be poor enough to cause the polymer to aggregate and form particles, but still good enough that the polymer chains on the particle surface are swollen, which prevents particle-particle aggregation during polymerization. In one embodiment, solvents used have Hildebrand solubility parameters about 4 to about 5 MPa^{1/2} above or below (i.e., more or less polar) than that of the forming polymer. For example, the formation of poly(divinylbenzene) (19.3 MPa^{1/2}) by precipitation polymerization can be performed in the solvent acetonitrile (24.3 MPa^{1/2}) and 20:80 MEK/heptane (15.9 MPa^{1/2}) to yield monodisperse microparticles according to the present disclosure. Precipitation polymerization can be used to form particles from reactive monomers (i.e., ones that allow later functionalization of the particle) such as methacrylic anhydride. Furthermore, in some embodiments the viscosity of the solvent is a further factor to consider in the selection of the solvent. A low viscosity solvent is preferred. In one embodiment, the solvent has a viscosity of less than about 0.5 cP at 20° C. The solvent used for precipitation polymerization should have a boiling point greater than the polymerization temperature (typically 60-70° C. for thermally initiated polymerization), and it should not substantially react with the monomers or initiator. In the case of reactive monomers like MeAn, nucleophilic solvents like water, alcohols or amines should be avoided. Particles may also be obtained from photoinitiated precipitation polymerization, which allows lower boiling solvents to be used. Examples of solvents suitable for the precipitation polymerization of the present disclosure include but are not limited to heptane, toluene, xylenes, methyl ethyl ketone (MEK), tetrahydrofuran (THF), acetonitrile, ethyl acetate, benzene, cyclohexane, chloroform, or mixtures thereof. In the case of photoinitiated polymerization, solvents such as acetone, diethyl ether, dichloromethane and pentane may be used.

Hydrogels required in cell applications are usually highly hydrated and soft, which correlates with a low degree of crosslinking within the gel. However, a low level of crosslinker during precipitation polymerization is associated with low particle yields.

The present invention describes the use of a reactive, temporary crosslinker during precipitation polymerization to increase particle yield to at least 30%, preferably at least 50%, and most preferably at least 70% compared to a typical yield of less than 20% for prior methods of precipitation polymerization with low crosslinker loadings. As importantly, after polymerization is complete, the reactive crosslinks are cleaved to enable particle swelling. At the same time, this conversion allows introduction of hydrophilic ionic groups and additional desired functional groups, through careful choice of cleavage reagent. A moderate amount of permanent (which may be a more slowly erodible) crosslinker (5-20 mol % relative to temporary crosslinker), is included in order to prevent complete dissolution of the particles upon post-polymerization modification. In summary, the inventors have surprisingly found comonomer/solvent combinations that allow use of precipitation polymerization to give particles in high yield, with cleavable crosslinks that can be readily functionalized to give facile access to highly hydrated, soft, narrow-disperse microgels.

It was found that the monomer methacrylic anhydride (MeAn), or its acrylic analog, acrylic anhydride, are particularly suitable for the present method as they can produce a polymer with anhydride groups that are easily functionalized, and because anhydride crosslinks can be easily cleaved to allow swelling of the as-formed, highly crosslinked particles into microgel particles.

MeAn is a divinyl monomer that undergoes two types of polymerization that consume both vinyl groups: crosslinking and cyclopolymerization (a non-crosslinking form of polymerization) (Scheme 1). Without wishing to be bound by theory, cyclopolymerization is a “linear” polymerization in that it does not lead to branching or crosslinking. In cyclopolymerization, the growing polymer chain adds to the two vinyl groups one after the other leading to the formation of a ring (5-membered or 6-membered in the case of MeAn) along a single polymer chain. Even though both vinyl groups are consumed, it is not a crosslink. While some divinyl monomers, like diallyldimethylammonium chloride, experience only cyclopolymerization, MeAn shows both types of reaction in a ratio that varies with experimental conditions (temperature, solvent, monomer concentration).⁸

Accordingly, there is provided a temporary crosslinker monomer of formula (I),(Ila) to (Ilf):

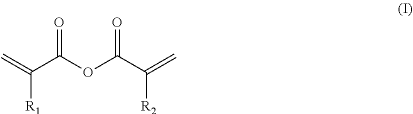

where R₁ and R₂ are independently selected from H, C₁-C₄ linear or branched carbon chain, benzyl, phenyl or OJ, where J is defined as a C₁-C₄ linear or branched carbon chain.

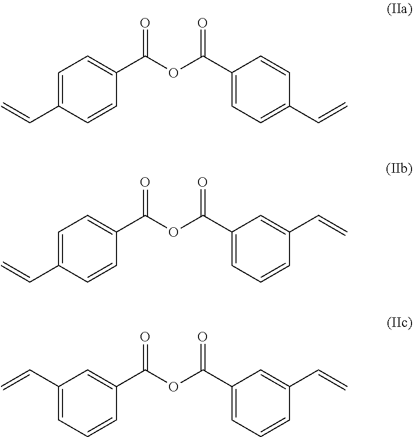

Symmetric as well as mixed anhydrides (as shown in formulas Ila to Ilf) are suitable temporary crosslinkers. Compound Ila is 4-vinylbenzoic anhydride, compound Ilb is 3,4-vinylbenzoic anhydride and compound IlC is 3-vinylbenzoic anhydride. All three formulas are suitable temporary crosslinkers, as are mixtures of different symmetric or mixed anhydrides.

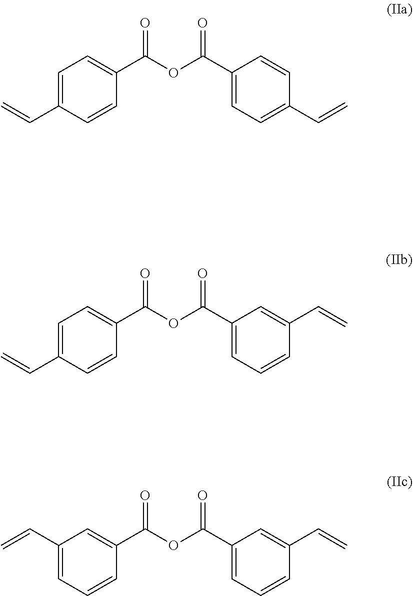

Where n is an integer from 1 to 3.

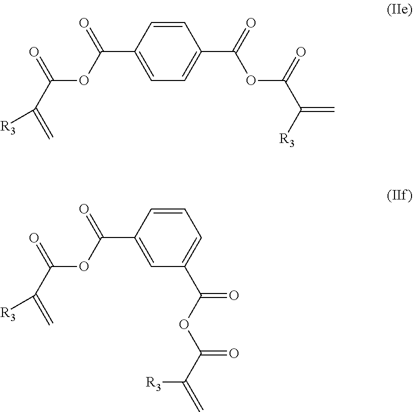

where R₃ is independently H or methyl.

Cyclic anhydrides such as maleic anhydride, citraconic anhydride, or itaconic anhydride are not suitable as temporary crosslinkers for the present method because they have only a single vinyl group and cannot be considered as crosslinkers. In addition, these compounds have poor polymerization efficiency under certain conditions, such as when present as more than 50 mol % of the monomer mixture, which limits their usefulness.

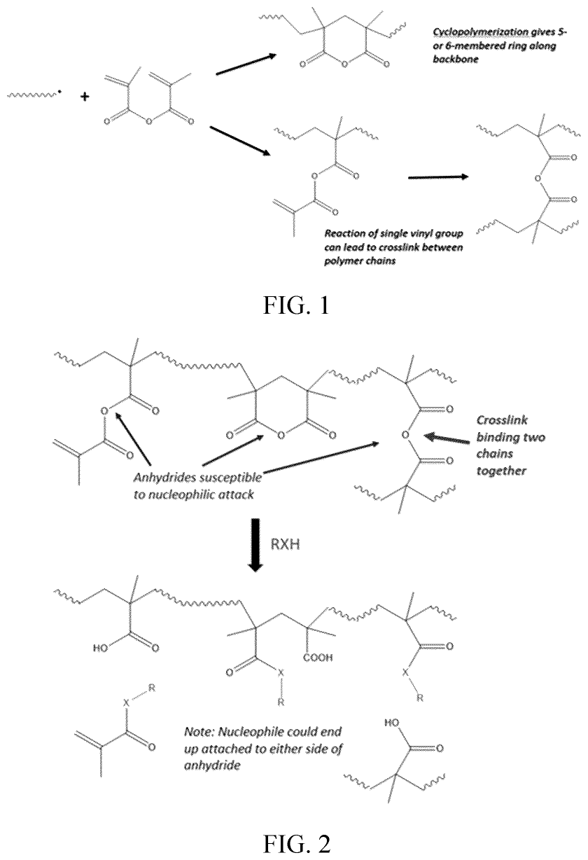

FIG. 1 illustrates polymerization of methacrylic anhydride showing cyclopolymerization, where the two vinyl groups are consumed in sequential reactions, or a more conventional reaction where only one vinyl group reacts. If the second vinyl group reacts ata later time, a temporary crosslink is formed.

The precipitated microparticles formed have a temporary crosslinker monomer of formula (Illa), (Illb), (Illc), (Illd), (Ille), (Illf), (Illg), (Illh), (Illi), (Illj), (Illk), (Illl), (Illm), (Illn), and/or (Illo):

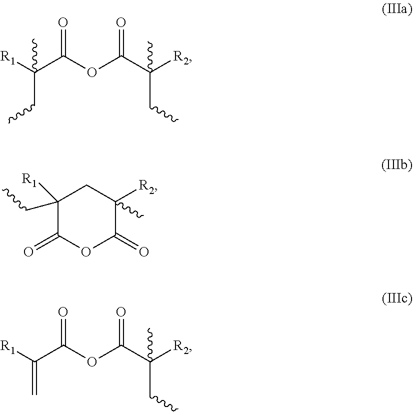

where R₁ and R₂ are independently selected from H, C₁-C₄ linear or branched carbon chain, benzyl, phenyl or OJ, and J is defined as a C₁-C₄ linear or branched carbon chain. The wavy lines represent the extended polymer backbone.

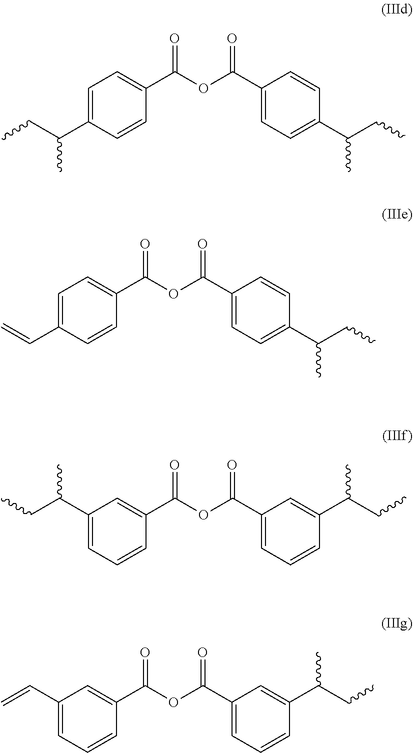

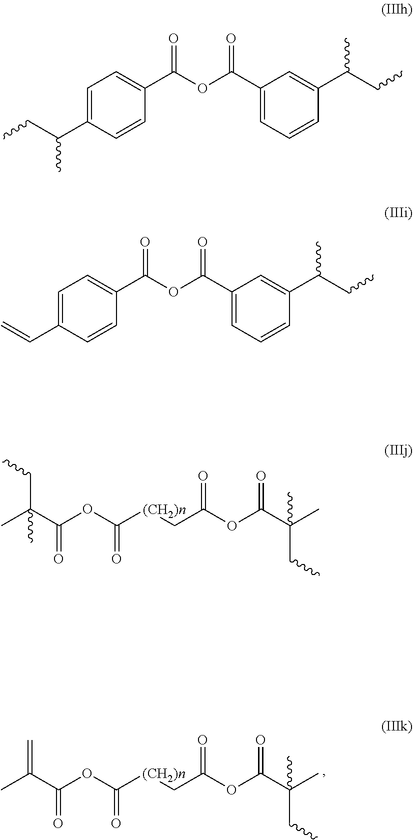

where n is an integer from 1 to 3,

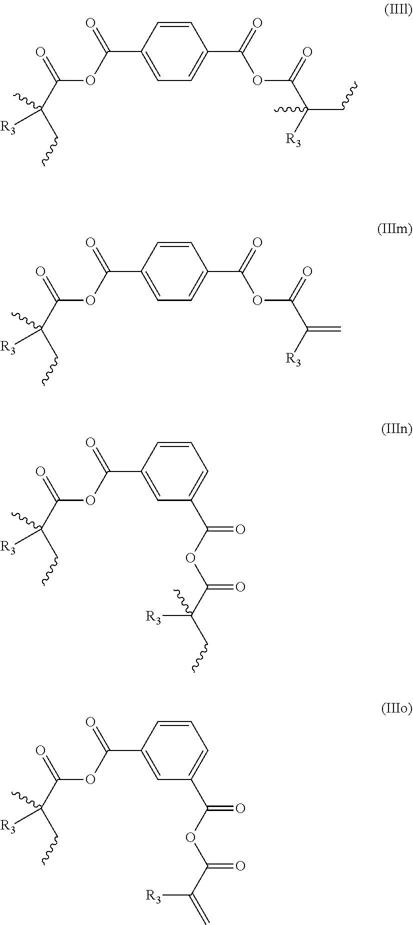

where R₃ is independently H or methyl.

These monomers can be functionalized into a multitude of monomers for example of formulas (IV) described below.

As illustrated in FIG. 2, the polymer will have MeAn groups where only one vinyl bond has reacted as well as those where both have been consumed, either by cyclopolymerization or by crosslinking. If the polymer is exposed to nucleophilic reagents such as water, alcohols, thiols or amines, the anhydride groups would be consumed leading to the formation of carboxylic acids, esters including thioesters, or amides. In the course of this reaction, the polymer would be functionalized and the anhydride crosslinks would be cleaved. If these were the only crosslinks, the particle would dissolve. For this reason, a conventional crosslinker such as ethylene glycol diacrylate, ethylene glycol dimethacrylate (EGDMA), diethyleneglycol diacrylate, diethyleneglycol dimethacrylate (DEGDMA), oligo(ethyleneglycol) diacrylate, oligo(ethyleneglycol) dimethacrylate, trimethylolpropane triacrylate, trimethylolpropane trimethacrylate, N,N′-methylenebisacrylamide (MBA), N,N′-methylene dimethacrylamide, polyvinyl or polyallyl ethers of glycol, of glycerol, of pentaerythritol, of carbohydrates; divinylbenzenes (DVB), trivinylbenzenes, divinylpyridines, or similar, are added as permanent crosslinkers in amounts between 1 and 30%, and preferably between 5 and 20% of total monomer weight, in order to ensure that the particles will survive functionalization and hydrolysis. The scheme below shows additional amine-functional permanent crosslinkers, where R and R₁=H or methyl, R′=alkyl or aryl, and x=1 or 2.

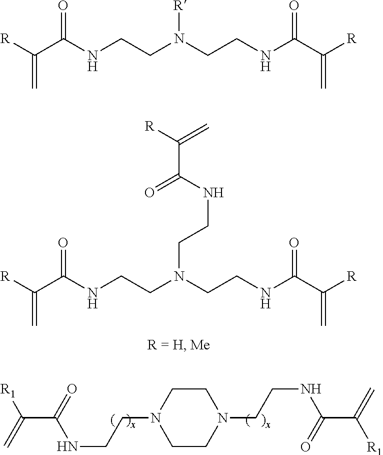

In cases where the microgel particles are required to undergo eventual spontaneous hydrolytic or enzyme-mediated degradation, the permanent crosslinker can be chosen from a group of known degradable crosslinkers containing a group cleavable under physiological conditions over a suitable time frame, including but not limited to disulfide groups, labile esters, labile acetals and ketals, and hindered anhydride groups. They all take the form of a degradable spacer between two monomer units. The degradation could occur in a number of ways including hydrolysis, enzymatic, redox or photochemical.

Examples of such slowly degradable crosslinkers include disulfide containing divinyl or higher vinyl crosslinkers such as bis(2-methacryloyl)oxyethyl disulfide or bis(2-acryloyl)oxyethyl disulfide, or the corresponding methacrylamides or acrylamides (R₁=Me, H; x=1-3)

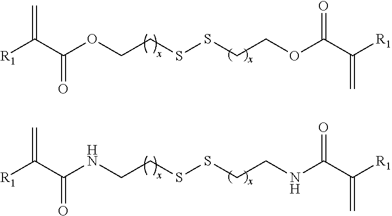

labile acetal-containing diacrylates or dimethacrylates such as b is[(2-methacryloyloxy)ethoxymethyl] ether

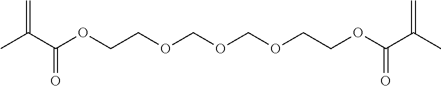 [SMILES: C=C(C)C(=O)OCCOCOCOCCOC(=O)C(=C)C]

or corresponding crosslinkers containing a single acetal unit where R₁=H, Me and R₂=H, alkyl (C₁-C₈, linear or branched), or aryl:

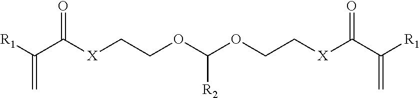

Other degradable crosslinkers include bis-methacrylate-terminated polylactic/glycolic acid oligomers and their multi-arm analogs, as well as acrylate and acrylamide analogs where R₁=H, Me; R₂=H, Me; R₃=H, Me:

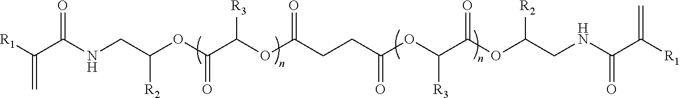

Further degradable crosslinkers may be based on ester linkages made degradable by proximity to an amine group, such as the two bisacryloyl amino esters shown below, as well as analogous crosslinkers that retain the acrylate ester based on the 2-aminoethanol motif.

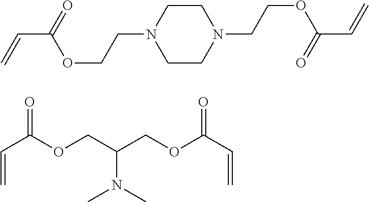

Furthermore, degradable crosslinkers may incorporate hydrolytically labile siloxane bonds such as the dimethyldi(methacryloyloxy-1-ethoxy)silane shown below, as well as analogous bis acrylate and bisacrylamide and bismethacrylamide crosslinkers, and analogous crosslinkers containing spacers longer than ethyl, multiple dialkyl siloxane labile units, and well as multi-arm analogs of the above.

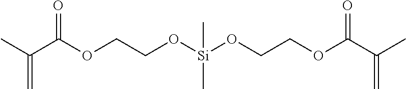 [SMILES: C=C(C)C(=O)OCCO[Si](C)(C)OCCOC(=O)C(=C)C]

Furthermore, degradable crosslinkers may contain labile O—N linkages such as in the N,O-dimethacryloylhydroxylamine shown below.

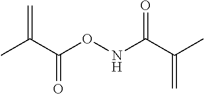

Finally, degradable crosslinkers may contain matrix metalloprotease (MMP)-cleavable groups such as (Pro-Leu-Gly-Leu-Trp-Ala) to allow matrix metalloproteases (MMP1, MMP3, MMP7 and MMP9) to degrade the polymer network.

FIG. 2 provides a reaction scheme showing reaction of methacrylic anhydride-based polymer with nucleophiles (RXH), which could take the form of hydrolysis or functionalization. Reaction of anhydride bridging two chains will lead to loss of crosslink. X is selected from NH, NR, O, and S, and R is a suitable biocompatible compound or molecule. For example, R can be defined as linear or branched C₁-C₁₈, aryl, heteroaromatic, saccharides, fluorophores, amino acids, peptides such as RGD, polymerization initiator, polyethylene glycol (PEG), betaines, proteins, ethylenediamine or other biomolecules (nucleotides, DNA, RNA, therapeutic molecules or agents and the like). The microparticles comprising the anhydride monomers of formulas (Illa) to (Illc) are functionalized to be monomers of formulas (IVa) to (IVc). Similar monomers are derived from formulas (Illd) and (Ille). The exemplary monomers shown below are derived from a hydrolysis and/or functionalization of the temporary crosslinker monomer of formula (Illa) to (Illc).

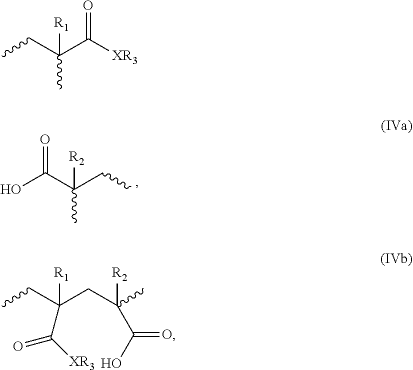

where R₁ and R₂ are H, alkyl (C₁-C₄), phenyl or benzyl, X is O, NH, NR₃, or S, and R₃ is H, alkyl (C₁-C₁₂), aryl, heteroaromatic, polyethylene glycol, saccharides, fluorophores, amino acids, peptides, other biomolecules (DNA, RNA, etc.), or other monomers.

The microparticles described in the present invention are formed by precipitation polymerization of specific mixtures of comonomers and crosslinkers under solvent conditions where the resulting polymer takes the shape of narrow-disperse or mono-disperse microspheres where the average diameter ranges from about 0.3 to about 20 micrometer, depending on the nature and amounts of the solvents, monomers and crosslinkers used.

Comonomers may be used with the crosslinkers in the precipitation polymerization process. In one embodiment, comonomers may be alkyl (C₁-C₁₂) methacrylates and acrylates, alkyl (C₄-C₁₂) methacrylamides and acrylamides, styrene, 3- or 4-alkylstyrenes where the alkyl may be linear or branched C₁-C₈, as well as styrenes carrying alkyl ether or alkylester substituents in the 3 and/or 4 position.

Comonomers may also be acrylic and methacrylic monomers that carry ethyleneglycol and methoxyethyleneglycol sidechains incorporating between 1 and 4 units, and optionally mixed length oligoethyleneglycol sidechains, as well as mixtures thereof.

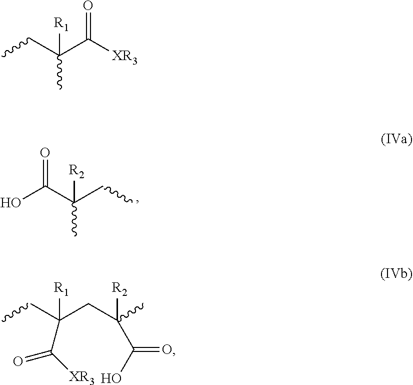

where R₁=H, alkyl (C₁-C₄); R₂ or R₃=H, alkyl (linear and branched, C₁-C₁₂), phenyl, benzyl, dialkylaminoethyl, dialkylaminopropyl, dialkylaminobutyl, alkoxyethyl, oligo(ethyleneglycol), methoxy oligo(ethyleneglycol).

Comonomers can also be vinylethers, linear and cyclic N-vinyl amides, or vinylesters, such as shown below,

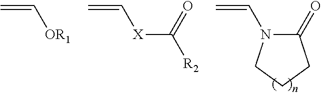

where R₁=alkyl (linear or branched, C₁-C₈); X=O or NH, R₂=H, alkyl, phenyl; n=1,2.

In one embodiment, the resulting composition based on dry weight includes microparticles having an average particle diameter (D) of between 0.3 and 20 micrometers where the coefficient of variation is less than 0.3, or more preferred less than 0.2, or most preferred less than 0.1. In one embodiment, the composition based on dry weight includes microparticles having an average particle diameter of between 0.3 and 20 micrometers where the coefficient of variation is less than about 0.3, less than about 0.2, or less than about 0.1 micrometers. In one embodiment, the expression “includes microparticles” in the context of a composition is defined as the composition comprising at least about 50%, at least about 60%, at least about 70%, at least about 80%, or at least about 90% microparticles by weight. The size of the particle can be selected based on the specific application whether the particle is desired to be internalized by cells or not, for example for a vaccine delivery a size of 0.1 to 1 micrometers is preferred and for cryopreservation or cell chaperones a size of 1 to 10 micrometers is preferred.

One aspect of the current invention is that these nano- and microparticles are formed in the presence of a high amount of a temporary crosslinker, which aids in the formation of high particle yields while enabling subsequent controlled decrosslinking and chemical modification by hydrolysis and introduction of desired functional groups to serve multiple applications described below. The functionalization renders the nano and microparticles biocompatible. The anhydride groups when contacted with an aqueous body fluid will react and reduce the pH and are thus not particularly biocompatible.

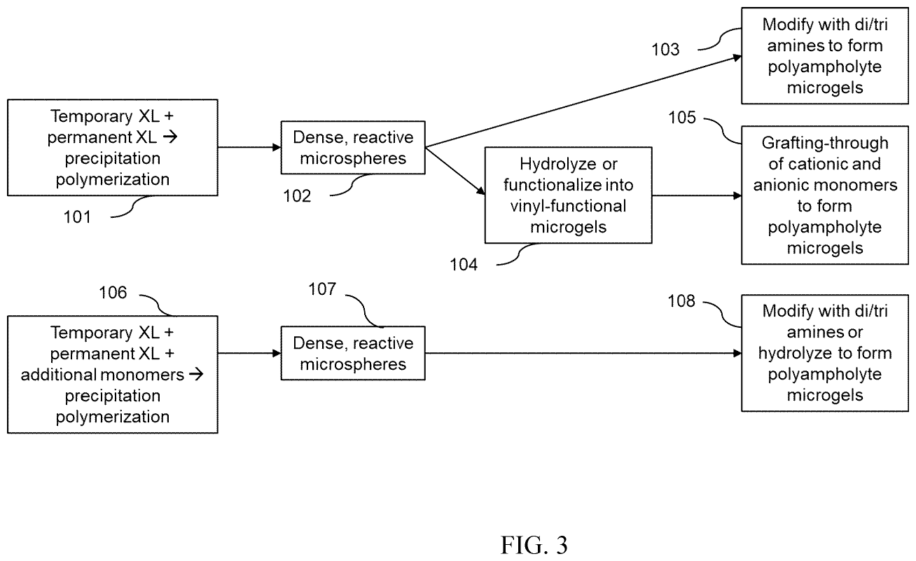

FIG. 3 shows three approaches to forming micron-range microgel particles with polyampholyte properties. All three approaches start with a precipitation polymerization on a temporary divinyl crosslinker (e.g., methacrylic anhydride, MeAn) together with a permanent crosslinker (e.g., diethyleneglycol dimethacrylate (DEGDMA). In the first approach, the temporary crosslinker (temporary XL) and the permanent crosslinker (permanent XL) undergo a precipitation polymerization 101 to yield dense, reactive microspheres 102. The dense, reactive microspheres are then modified 103 with di or tri amines to form polyampholyte gels. In the second approach, the dense reactive microspheres 102, are instead hydrolyzed or functionalized into vinyl-functional microgels 104. Then the grafting-through 105 of cationic and anionic monomers forms polyampholyte microgels. In the third approach, the temporary crosslinker, and the permanent crosslinker are combined with additional monomers in the precipitation polymerization 106, to yield dense reactive microspheres 107. The dense reactive microspheres 107 with the additional monomers are then modified 108 with di or tri amines or hydrolyzed to form a polyampholyte microgel.

In one embodiment, the microparticles formed from the precipitation polymerization consist or consist essentially of the temporary crosslinkers and the permanent crosslinkers according to the present disclosure. In a further embodiment, the microparticles consist or consist essentially of methacrylic anhydride and/or acrylic anhydride, and the permanent crosslinkers according to the present disclosure. In yet a further embodiment, the microparticles consist or consist essentially of methacrylic anhydride and/or acrylic anhydride as well as a permanent crosslinker selected from the group consisting of ethylene glycol diacrylate, ethylene glycol dimethacrylate (EGDMA), diethyleneglycol diacrylate, diethyleneglycol dimethacrylate (DEGDMA), oligo(ethyleneglycol) diacrylate, oligo(ethyleneglycol) dimethacrylate, trimethylolpropane triacrylate, trimethylolpropane trimethacrylate, N,N′-methylenebisacrylamide (MBA), N,N′-methylene dimethacrylamide, polyvinyl or polyallyl ethers of glycol, of glycerol, of pentaerythritol, of carbohydrates; divinylbenzenes (DVB), trivinylbenzenes, divinylpyridines, and combinations thereof. In an additional embodiment, the microparticles consist or consist essentially of methacrylic anhydride and/or acrylic anhydride and DEGDMA.

Without wishing to be bound by a theory, the formation of the microparticles is such that the density will be higher in the center versus the surface, and the microparticle is stiffer in the core versus the surface. The particles grow by the deposition of the newly formed polymer, which consists of lightly crosslinked or branched polymer chains. The divinyl temporary crosslinkers of the present disclosure provide more available double bonds for crosslinking and as the particle grows there are more available double bonds to maintain the growth than a monovinyl monomer. Material that is captured early in the particle growth, and is hence close to the particle core, will undergo further crosslinking reactions such that it becomes denser and stiffer. The radial gradient may be further enhanced by using crosslinkers that are preferentially incorporated. For example, if a small amount of electron-rich crosslinker such as divinylbenzene or a divinylether is added to the MeAn/DEGDMA polymerization, the electron-rich crosslinker is incorporated preferentially in the core due to its higher reactivity compared to the other monomers, both of which are electron-poor. This results in a higher level of permanent, electron-rich crosslinker in the particle core than at the surface. These properties are inherent to the precipitation polymerization method used herein, as the sequential radial growth of the particles leads to the surface composition at any point reflecting the composition of the copolymer formed at that time.

In one example, a cleavable temporary crosslinker such as methacrylic anhydride (MeAn) is copolymerized with a permanent crosslinker such as diethyleneglycol dimethacrylate (DEGDMA) at 5% (weight/volume) total monomer loading in a solvent mixture comprising 60 vol % of methyl ethyl ketone and 40 vol % n-heptane, and in presence of about 2 wt % of Al BN (relative to total monomer) serving as free radical initiator. The polymerization is carried out in 20 mL screwcap glass vials, which are heated to a temperature of 55 to 80° C., and preferably 65 to 75° C. for 4 to 24 hours, and preferably 12 to 20 hours. As known in the art, there exist thermal initiators that can initiate polymerization at lower as well as at higher temperatures. As well, there are known in the art radical initiators based on redox processes that can initiate polymerization at these and other temperatures, that are incorporated herein.

The ratio of temporary crosslinker to permanent crosslinker may range from 50:50 to 99:1 mol percent, and preferably from 80:20 to 95:5. Examples of temporary, reactive crosslinkers (formulas (I) and (II)) include MeAn, acrylic anhydride, and 4-vinylbenzoic anhydride. Examples of permanent crosslinkers include EGDMA, DEGDMA, methylenebisacrylamide, divinylbenzene, and the like.

In this example, the resulting microspheres are formed in high yield (67±10%) as all monomers present during the precipitation polymerization are divinyl compounds leading to a higher degree of crosslinking and hence more efficient particle formation. This feature significantly increases the isolated yield of these microspheres (40% to 80%) over that of comparable particles formed by precipitation copolymerization in the absence of the temporary crosslinker but with the same amounts of permanent crosslinkers. In a specific example, the yield obtained by a precipitation polymerization using a ratio of 10:90 of permanent crosslinker to temporary crosslinker according to the present disclosure is higher than the yield obtained by precipitation polymerization using a permanent cross linker to simple monovinyl compounds (non-crosslinkers) ratio of about 10:90.

In some embodiments an initiator, preferably a photoinitiator, can be used to decouple the rate of initiation from the reaction temperature. The rate of polymerization and polymer radial distribution are both affected by the reaction temperature. The use of a photoinitatior rather than thermoinitiator allows for a constant rate of initiation over a range of reaction temperatures. The photoinitiator can be selected such that the wavelength of the photo stimulus is not absorbed by the solvent or the monomers. For example, 2,2′-Azobis(2-methylpropionitrile) (AIBN) can be used to initiate the precipitation polymerization following irradiation with light having a 365 nm wavelength.

### Cryopreservation

Swollen and suspended microgels having properties described herein may be combined with mammalian and other cells in culture, suitably, in a 10,000:1 to 1:1, and preferably 5000:1 to 200:1 volume ratio of microgels to cells. In some embodiments, the microgels form jammed gels upon co-sedimentation with cells. Jammed gels are close-packed arrays of soft particles that are solid- or gel-like under low stress, but can flow under higher stress. Entrapment of cells within the jammed gel can mitigate cryodamage to cells by reducing ice crystallization around the cells, and by partial dehydration of the cells. In another embodiment solutions or suspensions of highly swollen microgels form viscous solutions that prevent cell sedimentation. In some embodiments, the microgels used for cryopreservation have an anionic:cationic ratio of between 70:30-30:70. In some embodiments, the hydrogel microparticles are in a concentration of 1-25 wt/v %. This value can depend on the microgel stiffness. Stiffer microgels capable of co-sedimenting with cells to form a jammed gel can be used effectively in the range of 1-5 wt/v % and softer microgels capable of forming a volume filling viscous solution that prevents cell sedimentation at a concentration range of 5-25 wt/v % that can be separated from cells by centrifugation.

During freezing and thawing, these microgels surround the cells, and prevent ice crystals from causing cell damage by penetrating cell walls. At the same time, slow freezing of the continuous media will lead to increased osmotic pressure in the microgels, which in turn will lead to partial dehydration of the cytosol. The resulting higher osmotic strength (higher protein concentration) within the cytosol will reduce ice crystal formation within the cells. Additionally, the microgels can reduce ice crystal recrystallization under thawing conditions that would produce larger, cell-damaging ice crystals. In another embodiment, the addition of microgels under rapid freezing conditions leads to the vitrification of the continuous media inhibiting ice crystal formation.

These microgels, by virtue of their relatively large size (1-10 micrometer) and non-fouling nature, are not likely to be taken up into the mammalian cells by affinity-mediated processes or even pinocytosis, and hence overcome key concerns with current cell cryoprotective agents such as dimethyl sulfoxide (DMSO) (used with cells including stem cells), and ethyleneglycol/glycerin and other sugar-derived molecules⁹ (used with cryostored blood), that is, residual cytotoxicity and the effect of the cryoprotective agents on the cell's ability to differentiate (stemness) as well as time needed to remove intra-cellular cryoprotective agents.

Mono-disperse microgel particles enable better control over degree of deformation of cells into interstitial volumes between microgels. Moreover, in some embodiments the soft, deformable polyampholyte microgels of the present disclosure can be used to replace conventional cryo-protective agents that are cell-penetrating such as DMSO. This is particularly advantageous for cells that are sensitive to cryo-protecting cell penetrative agents (e.g., DMSO). The polyampholyte microgels of the present disclosure can prevent rapid cell sedimentation to ensure cell-survival during freeze-thaw processes encountered during cryo-storage. Avoiding the formation of external ice crystals is important as these ice crystals can pierce cells membranes. Advantageously, external ice crystals can be minimized or avoided with the present microgel. Furthermore, the microgel of the present disclosure can also dehydrate the cytosol, thereby preventing cell damage by ice crystal formation within cells.

These cell-sized hydrogel particles are much less likely to be taken up by the cells than would be linear polymers of similar composition, reducing concerns such as cytotoxicity or interference with cellular differentiation. In addition, the microgels of the present disclosure are formed with polymers that are sufficiently cross-linked to minimize or prevent cell uptake.

Methods of Making Non-Penetrating Cell Cryoprotective Agents

Microspheres as described herein may be turned into cryoprotective hydrogel microparticles by three methods:

Post-modification of the as-formed microspheres by treatment with a slight excess of N,N-dimethylethylenediamine, 3-(dimethylamino)propylamine, N,N-dimethylaminoethanol, choline, amino acids, betaines, diamines, triamines, polyamines or other similar compounds that convert anhydride groups into a combination of a free carboxylic acid and an amide or ester carrying an amine group. This modification essentially converts the temporary anhydride crosslinks as well as cyclic anhydrides formed during the precipitation polymerization, into a mixture of anionic carboxylates and cationic groups that confer polyampholytic character to the resulting microgels. As a result of the cleavage of the temporary anhydride crosslinker, and depending on the amount of permanent crosslinker present, these microgels are now highly swellable and deformable, with overall moduli approaching those of mammalian cells and tissues. These particles have no or very minimal extraneous surface residues of stabilizer. These microgels have shown utility as non-cell penetrating cell cryoprotecting agents.

An alternate method for forming mono-disperse, cryoprotective microgels involves grafting a polyampholyte onto the hydrogel microparticles using pendant vinyl groups. The pendant vinyl groups may be residual vinyl groups of the permanent crosslinker used in the precipitation polymerization (e.g., DEGDMA), or they may be vinyl groups added by functionalization of the reactive microparticles with, for example, 3-aminopropylmethacrylamide, 2-aminoethyl methacrylate, or 2-hydroxyethyl methacrylate. Hydrolysis, or functionalization followed by hydrolysis, of the as-formed microspheres under mildly alkaline conditions, converting the temporary anhydride crosslinks as well as cyclic anhydride groups and pendant anhydrides into carboxylic acids, and, in the case of functionalization, amides or esters (functionalized carboxylic acids). Subsequently, the now highly swollen microgels can be modified into polyampholyte microgels by a process called grafting-through, wherein the hydrolyzed microgels are suspended in aqueous mixtures of anionic and cationic monomers including but not limited to, methacrylic acid (MAA) and N,N-dimethylaminoethyl methacrylate (DMAEMA), together with a water-soluble free radical initiator, and heated, or alternatively irradiated with light, such that the resulting copolymerization leads to copolymers of the water-soluble monomers covalently attached by grafting-through the pendant vinyl groups. Examples of anionic, cationic, and zwitterionic monomers are provided below:
Anionic—Acrylic acid, Methacrylic acid, 2-carboxyethyl acrylate, 2-acrylamido-2-methylpropanesulfonic acid (or sodium salt), vinylsulfonic acid, styrenesulfonic acid (or sodium salt), phosphonic acids.
Cationic—N,N-dimethylaminoethyl methacrylate, N,N-dimethylaminoethyl acrylate, 3-(N,N-dimethylamino)propylmethacrylamide, 3-aminopropylmethacrylamide, 2-(methacryloyloxyethyl)trimethylammonium chloride, 3-(methacrylamidopropyl)trimethylammonium chloride (the preceding monomers are represented in the general structures shown below), and vinylpyridine.

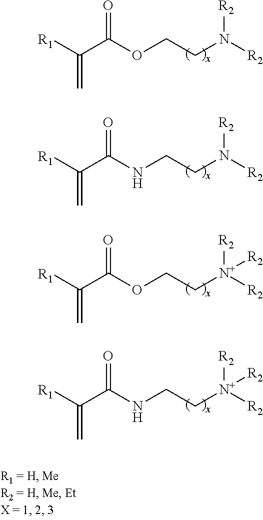

Zwitterionic—2-Methacryloyloxyethyl phosphorylcholine, N-(2-methacryloyloxy)ethyl-N,N-dimethylammonio propanesulfonate, N-(3-methacryloylimino)propyl-N,N-dimethylammonio propanesulfonate, 3-(2′-vinyl-pyridinio)propanesulfonate, 3-[[2-(methacryloyloxy)ethyl]-dimethylammonio]propionate, as well as other betaine monomers.

Finally, the as formed microgels may be modified into non-penetrating cryoprotective microgels by hydrolysis followed by absorption of polycations or copolymers having net positive charge. Examples may include homopolymers of permanent cationic monomers such as aminoethylmethacrylamide, dimethylaminoethyl methacrylate, and similar cationic monomers, homopolymers of dimethylaminoethyl acrylate and similar charge-shifting monomers, as well as copolymers of such permanent or charge-shifting monomers with other cationic, neutral, anionic or hydrophobic monomers described elsewhere in this filing, provided the resulting copolymers have a net cationic charge, and comprising 30 to 99 mol % cationic monomers, preferably 50 to 80% cationic monomers, and most preferably 60-70% cationic monomers.

Provided the above final microgels have proper ratios of anionic and cationic groups, which includes ratios ranging from 80:20 to 20:80 anionic to cationic, and preferably 70:30 to 40:60 anionic to cationic, combinations of these microgels with (mammalian) cells in the form of dense suspensions, with swollen microgel to cell volume ratios in the range of 10,000:1 to 1:1, and preferably 5000:1 to 200:1, have the ability to change the freezing behavior of water around these cells such as to prevent damage to these cells during long-term storage under cryogenic conditions. These microgels may optionally include neutral hydrophilic and hydrophobic groups, and these as well as the charged groups may be introduced during the original precipitation polymerization or during post-functionalization with small molecules, or during post-grafting through polymerization, or post-hydrolysis absorption of predominantly cationic copolymers. Examples of neutral hydrophilic and hydrophobic monomers are provided below:
Neutral Hydrophilic Functionalizing Reagents—aminoethanol, PEG-amine, ethyleneglycol, and glycine methyl ester, (and other amino acids).
Neutral Hydrophilic Monomers—2-hydroxyethyl methacrylate, 2-hydroxyethyl acrylate, acrylamide, methacrylamide, N,N-dimethylacrylamide, N,N-diethylacrylamide, N-isopropylacrylamide, (and other acrylamides/methacrylamides), and PEG methacrylate.
Neutral Hydrophobic Functionalizing Agents—alkyl (C4-C12) and aryl amines, alcohols (C2-C12), and thiols (C2-C12).
Neutral Hydrophobic Monomers—alkyl (C1-C12) methacrylates and acrylates, alkyl (C4-C12) methacrylamides and acrylamides, styrene, and 4-methylstyrene.

A key benefit of the present microparticles and methods are that the microgels formed, by virtue of their size and non-fouling compositions, are highly unlikely to enter into the cytosol of the cryoprotected mammalian cells, obviating a key concern about use of penetrating cryoprotective agents commonly used including ethylene glycol, propylene glycol, and especially dimethylsulfoxide (DMSO), a known sensitizer and cytotoxic molecule that has been found to affect the ability of stem cells to differentiate.

Further, these microparticles are predicted to be less susceptible to cellular penetration than linear copolymers having similar compositions.

Other benefits are that these microgels, when formed by precipitation polymerization methods described herein, are formed without need for surfactants or steric stabilizer and thus have surfaces free or substantially free from these potentially interfering compounds.

As well, the continuous growth of the particles over the course of the precipitation copolymerization means that the outer shell is more lightly crosslinked, and hence softer, than the particle core.

Furthermore, these microgel particles may be separated from the cells after thawing from cryostorage, by simple centrifugation based on their different rates of sedimentation relative to cells. Alternatively, the microgels may also be separated from cells by filtration, in cases where microgels are significantly smaller than cells. Examples include microgels that are 0.5 to 3 micrometer in diameter, compared to typical cell diameters of 10 to 15 micrometer.

As well, separation of these microgels from cells after cryostorage and thawing can be facilitated by incorporating magnetic nanoparticles within the microgel particles, either by entrapment of preformed magnetic nanoparticles during precipitation polymerization, or by formation of magnetic nanoparticles within the anionic hydrolyzed microgel particles prior to incorporation of cationic charges, using methods for forming such magnetic nanoparticles within anionic polymer gels that are known in the art. These methods include absorption of soluble iron salts followed by their precipitation into insoluble, magnetic iron oxide nanoparticles.

Finally, the described process of precipitation polymerization involving high levels of temporary crosslinker leads to high yields of soft, well-hydrated and mono- or narrow-disperse microgels that allow excellent packing around dispersed cells.

### Cell Mimetics

Also provided herein are analogous nano- and microparticles that are modified to serve as synthetic granular components of ECM for cells in different forms of cell culture, in particular, but not limited to, cell encapsulation for research or therapeutic purposes.

Such microgels may be used as cell mimetics in cell culture of adherent cells, where they can be used as granular components of synthetic composite organoids that comprise cells and microgels in ratios of 1:100 to 1:1, and preferably 1:20 to 1:3.

Such composites can provide many of the benefits to cells offered by real organoids consisting solely of cells, including attachment, without additional nutrient and oxygen demand. In fact, the presence of these permeable microgels can increase nutrient and oxygen supply to cells located within the composite cluster, as the connected networks of highly swollen, permeable microgels can act as diffusion paths for oxygen and nutrients, as well as efflux of cell products of low to moderate molecular weight, such as insulin.

In addition to helping maintain higher oxygen partial pressure and nutrient levels near the therapeutic cells, such artificial diffusion paths/networks of permeable microgels also have the potential to increase soluble signal transduction into and out of the cluster, and thus increase the kinetics of feedback-controlled systems such as glucose-triggered insulin response.

In reaggregates of islet cells or beta cells or other therapeutic cells with such microgel-based cell mimetics, these diffusion paths can take the place of the extensive microvasculature typically present in Islets of Langerhans.

These benefits hold especially for functional therapeutic cells and organoids encapsulated into semi-permeable hydrogel capsules for transplantation into humans as part of cell-based therapies for endocrine disorders such as diabetes, hemophilia, and lysosomal storage disorders.

Granular ECM Components within Capsules

Similar microgels to those as described above for use in cryopreservation, optionally additionally modified with cell attachment groups such as RGD (Arginine-Glycine-Aspartic acid), may be used as granular ECMs in different types of cell culture applications. These include co-encapsulation with donor or stem-cell derived mammalian endocrine cells designed for cell-therapies for endocrine disorders such as diabetes, Parkinson's, hemophilia, and lysosomal storage disorders.

They may also include cell cultures used to study cell behaviors in simulated tissue, including cancer cell migration through tissue during metastasis, immune cell migration as part of natural immune surveillance within tissue, cross-migration of embryonic and maternal cells during placenta formation in pregnancy, and spread of, e.g., bacterial infections within tissue.

The post-modifications can be designed to introduce chemical properties that enable use of the microgel particles in a number of biomaterials applications.

Suitable microgels may be formed for example by precipitation polymerization of methacrylic anhydride with diethyleneglycol dimethacrylate (DEGDMA) in a 90:10 mole ratio (99:1 to 80:20 with 95:5 to 85:15 preferred) at a total monomer loading of 5 wt % (1-20% with 2-10 wt % preferred), and in the presence of 2 weight % AIBN, in methyl ethyl ketone/heptane mixtures (60:40).

The resulting particles may be modified by hydrolysis or by functionalization with various modifiers, including amines, alcohols and thiols bearing hydrophobic, hydrophilic or biologically active groups. More specifically, these modifiers may be primary amines such as ammonia, or alkylamines where alkyl can be methyl, ethyl, propyl, butyl, etc, glucosamine, ethanolamine.

The above microgels may be post-modified with a cellular adhesion molecule, which, as used herein, can include all protein sequences capable of binding to an integral membrane protein (e.g., an integrin) on a cell, resulting in a cell-protein adhesion. As used herein, the terms “Arg-Gly-Asp” peptide or sequence or “RGD” peptide or sequence refer to a peptide or amino acid sequence having at least one Arg-Gly-Asp-containing sequence which can function as a binding site for an integrin type receptor as well as any functional equivalents.

The above microgels may be added to a suspension of mammalian (therapeutic) cells in sodium alginate or similar gel former, and dropped into calcium chloride for gelation.

They may also be added to cells deposited in other cell culture devices including multi-well plates, to moderate cell-cell interactions.

### Vaccine Applications

In another embodiment, there is provided nano- and microparticles, modified to serve as delivery platforms for antigens in a vaccine context. These nano- and microparticles may:
Act as carriers for the antigens, e.g., RNA including m-RNA, DNA, proteins, viral shell fragments, whole inactivated viral shells, or active innocuous viruses such as adenoviruses that have been modified to express the desired antigen protein.
Have cationic groups that can electrostatically bind the antigen during storage and administration to the recipient's immune system.
Have adjuvant properties to ensure recognition and processing by the host immune system, including where said adjuvant properties are based on cationic or polycationic groups, or on certain carbohydrate groups.
Have compositions where cationic or polycationic groups can be cleaved by spontaneous or enzyme-mediated hydrolysis in order to release the bound antigen payload over a timeframe beneficial to evoking a strong immune response in the recipient.
Have crosslinkers that may undergo slow spontaneous or enzyme-mediated hydrolysis in order to ensure ultimate clearance of the microparticles from the recipient through processes including renal clearance.
Have compositions including cationic and polycations groups as well as non-stoichiometric polyampholytes that can bind antigen for storage at room temperature defined as up to 40° C., and without need for cold-chain logistics during storage and transportation.
May additionally be loaded with silver nanoparticles to enhance cellular immune response, either by coprecipitation during precipitation polymerization, or by reductive precipitation from silver salts as part of the post-functionalization, or by adsorption of preformed silver nanoparticles onto the described polymer nanoparticles.

### Methods of Making Vaccine Delivery Particles

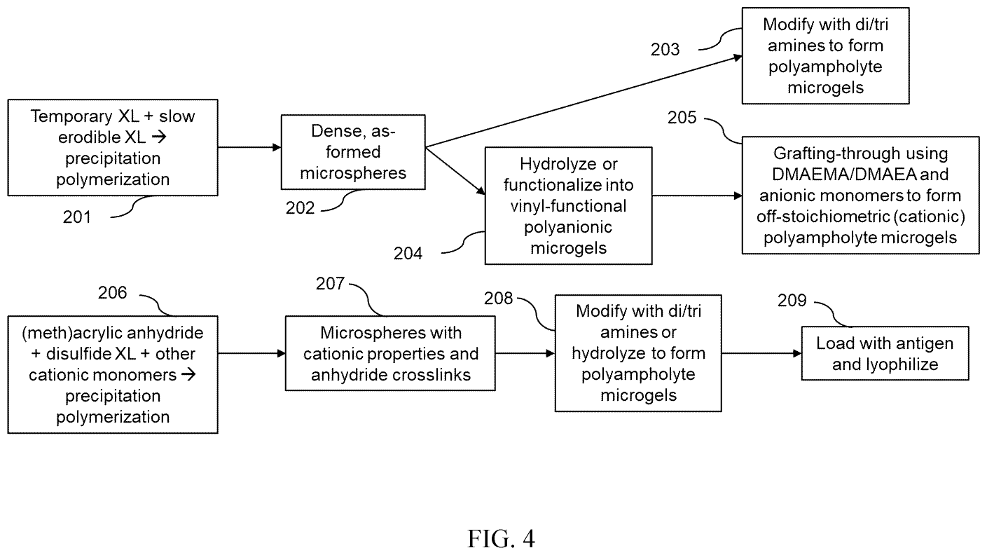

FIG. 4 shows two approaches to forming nanoparticles for use as antigen carriers suitable for vaccine applications. Both include an initial precipitation polymerization 201, 206 of a temporary crosslinker (e.g., methacrylic anhydride), together with a slowly erodible divinyl crosslinker (e.g., a disulfide-bridged dimethacrylate) to ensure the particles will ultimately be removed by renal clearance. The second approach includes the addition of cationic monomers in the precipitation polymerization 206. In the first approach, a dense, as-formed microsphere is obtained 202 which can then be modified with di or tri amines 203 or alternatively hydrolyzed or functionalized into vinyl-functional polyanionic microgels 204 which are then grafted-through 205 using DMAEMA/DMAEA and anionic monomers to form off-stoichiometric (cationic) polyampholyte microgels. In the second approach, microspheres with cationic properties and anhydride crosslinks 207 are obtained. They are then modified with di or tri amines 208, and may then be loaded with antigen and lyophilized 209.

In one example, submicron (0.1-0.9 micron) particles grafted with suitable copolymer may be used as vaccine delivery vehicles. Here, antigens based on proteins, mRNA, DNA or virus or bacterial shell fragments or whole inactivated virus or bacteria or other pathogens, may be absorbed into or bound to hydrogel particles post-functionalized with groups or polymer able to bind these antigens. This may involve cationic modifying groups or off-stoichiometric polyampholytes containing an excess of cationic charges in the grafted copolymer.

In addition to binding the antigen, the cationic nature of the microgels may assist in uptake of the antigen-loaded vaccine particles into macrophages or other cells upon administration.

As well, the cationic nature of the microgel surface may exert an adjuvant response upon introduction into tissue, either by injection, nasal administration, or other form of administration.

Alternatively, or in addition, silver nanoparticles may be introduced into the vaccine particles in order to enhance adjuvancy.

As well, the cationic groups or copolymers may be designed to undergo charge shifting into anionic groups, thereby releasing the antigen over a time frame suitable to elicit a sustained immune response.

As well, the copolymers grafted on to the microgels can be designed to bind the antigen in a fashion to prevent denaturation or other forms of deactivation during extended storage at elevated temperature, which may mean temperatures above −80° C., including storage at −30° C., −10° C., +2-8° C., or room temperature (defined as temperatures up to 37° C. or 40° C.) In particular, complexation of native proteins by certain synthetic or natural macromolecules can protect the protein from denaturation. Examples include the natural proteins called HERO proteins described in 2020, 10 and well as other Inherently Disordered Proteins (IDPs).11 As well, synthetic polyampholytes can sequester native proteins and protect them from denaturation during heating or dry storage. The copolymers grafted onto the present microgels can be designed to form a coacervate phase with the anionic microgel at physiological pH that can sequester native proteins and prevent denaturation during dry storage after lyophilization (freeze drying). A coacervate is defined as an electrostatically maintained, highly hydrated polymer phase comprising either a single polymer containing a near-stoichiometric balance of anionic and cationic charges (simple coacervate), or a pair or larger set of polymers and copolymers carrying a net stoichiometric or near stoichiometric balance of cationic and anionic monomers (complex coacervate). Alternatively, the microgel together with the grafted copolymers may form a complex coacervate phase upon complexation with a predominantly anionically charged protein or other antigen such as RNA or DNA, that similarly can protect the payload protein of polynucleotide from denaturation or other degradation upon storage. Finally, the antigen-binding coacervate may be produced by electrostatically absorbing a predominantly cationic polymer or copolymer into the anionic hydrolyzed microgel particle.

In all these cases, the net charge of the coacervate phase should be neutral or preferably cationic in order to promote absorption of the antigen, cellular uptake of the microgel particle, and adjuvancy. As well, the permanent crosslinker may be designed to also degrade over a time range suitable to enable ultimate renal clearance of injected vaccine particles.

### EXAMPLES

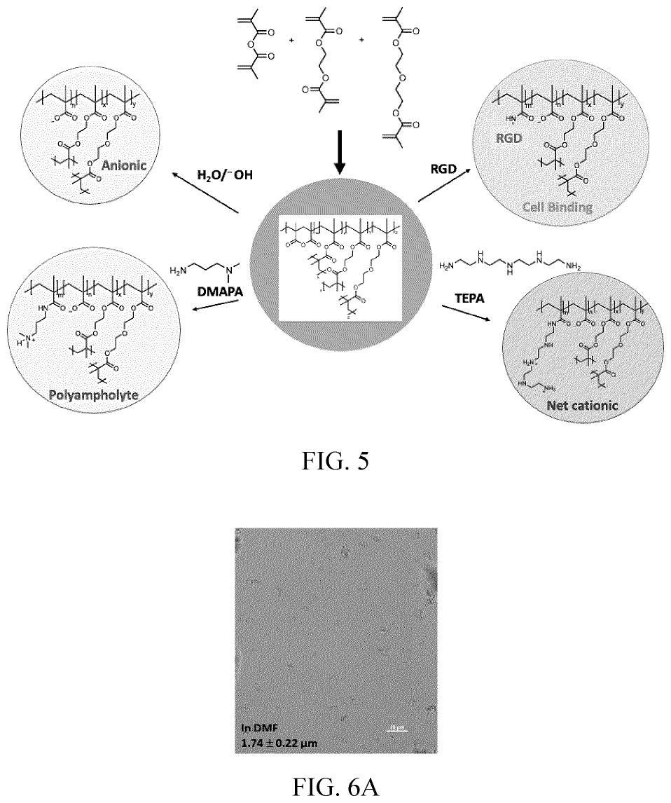

The following examples show the preparation of reactive particles based on MeAn, conversion of the reactive particles to a variety of functionalized hydrogel particles, and several applications of the hydrogel particles. Preparation of the reactive particles, and their conversion to hydrogels is shown schematically in FIG. 5.

All materials were used as received unless otherwise stated. 2,2′-Azobis(2-methylpropionitrile) (AIBN, 99.9%) was purchased from Dupont. Methacrylic anhydride (MeAn, 94%), ethylene glycol dimethacrylate (EGDMA, 98%), diethylene glycol dimethacrylate (DEGDMA, 95%), dimethoxy propane (DMPA, 98%), 2-hydroxyethyl methacrylate (HEMA,≥99%), p-toluenesulfonic acid monohydrate (pTSA,≥98.5%), 4-methoxyphenol (MEHQ, 99%), silica gel (technical grade, pore size 60 Å, 230-400 mesh), sand (50-70 mesh), potassium carbonate (anhydrous, 99%), 3-(dimethylamino)-1-propylamine (DMAPA, 99%), acetonitrile (ACN,≥99.5%), methyl ethyl ketone (MEK,≥99.0%), heptane (99%), acetone (≥99.5%), hexanes (≥99.5%), ethyl acetate (≥99.5%), N,N-dimethylformamide (DMF,≥99.8%), and chloroform-D (CDCl₃, 99.8% D) were purchased from Sigma Aldrich. Sodium chloride (NaCl, ACS reagent) and sodium hydroxide (NaOH, ACS reagent) were purchased from ACP chemicals. Disodium hydrogen diphosphate heptahydrate (Na₂HPO₄·7H₂O), sodium bicarbonate (NaHCO₃), hydrochloride acid 35-37 wt % (HCl, Reagent), glacial acetic acid (reagent grade), and sodium acetate (reagent grade) were purchased from Caledon Laboratories Ltd. Sodium dihydrogen orthophosphate (NaH₂PO₄·H₂O, Assured grade) was purchased from BDH Chemicals. Deuterium oxide (D₂O, 99.9% D) was purchased from Cambridge Isotope Laboratories Inc. Trypan blue 0.4%, phosphate buffered saline (PBS), Dulbecco's modified Eagle medium (DMEM, 4.5 g/L D-glucose, L-glutamine, 110 mg/L sodium pyruvate), 0.5% Trypsin-EDTA (10×), Penicillin-Streptomycin, bovine calf serum (BCS), and tetramethylrhodamine cadaverine, 5-(and-6)-((N-(5-aminopentyl)amino)carbonyl) tetramethylrhodamine (mixed isomers) (TAMRA cadaverine) were purchased from Thermo Fisher Scientific. 2-Propanol (certified ACS) and dimethyl sulfoxide (DMSO,≥99.7%) were purchased from Fisher Chemicals. RGD (Arg-Gly-Asp) (>95%) was purchased from Abcam.

Particles as-formed and/or after hydrolysis and/or functionalization were characterized by microscopy, ¹H NMR, and zeta potential measurements. Brightfield images were taken with a Nikon Eclipse LV100ND upright microscope, or a Nikon Ti Eclipse inverted microscope. Confocal images were taken with a Nikon Al Confocal Ti Eclipse microscope. Microgel diameters were measured manually with a 2-point measurement on brightfield images using Nikon NIS-elements Advanced Research software. ¹H NMR analysis was done with a Bruker 600 MHz for particles swollen in D₂O or DMSO-d₆. Zeta potentials were measured using a Zetasizer Nano ZS from Malvern.

### Example 1

The following example shows the synthesis of MeAn-containing particles with thermal-and photoinitiated polymerization. Precipitation polymerizations according to the scheme below were performed to obtain microparticles. The scheme shows conditions for a photoinitiated polymerization, but the same solution can be used for thermally initiated polymerization by heating at 60-70° C. In this and following examples, the MeAn-based particles will be denoted as (MED-X/Y/Z) where X represents the volume percentage of MEK in the MEK/Heptane co-solvent mixture used, and Y and Z represent the mol percentages of the two permanent crosslinkers, EGDMA and DEGDMA, in the total monomer pool, with the remaining amount representing MeAn

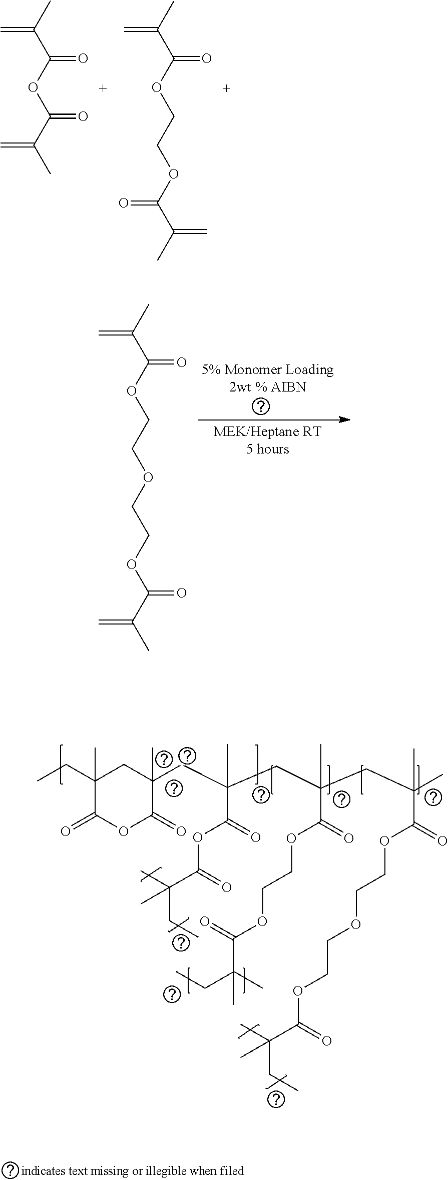

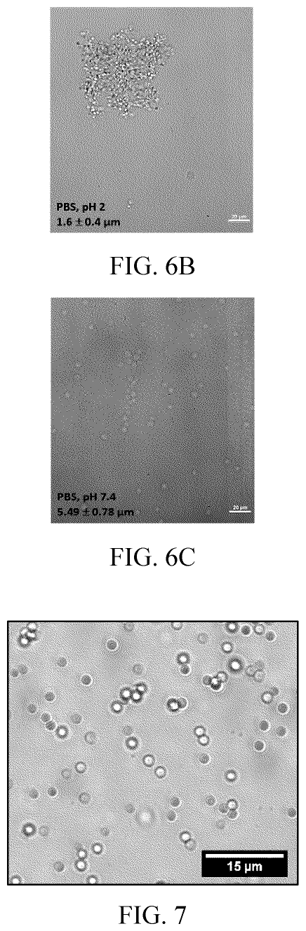

Polymerizations were typically done at a 5% (w/v) total monomer loading with 2 wt % AIBN relative to total monomer. For example, MED-55/5/5 particles were made from MeAn (1.596 g, 10.3 mmol), EGDMA (0.114 g, 0.57 mmol), DEGDMA (0.139 g, 0.57 mmol) and AIBN (0.037 g) dissolved in 35.15 mL of a 55/45 (v/v) MEK/heptane mixture (19.33 mL MEK, 15.82 mL heptane). The mixed solvents were prepared by weight using room temperature densities of 0.805 g/mL for MEK and 0.684 g/mL for heptane, such that 15.56 g MEK and 10.82 g heptane were combined to make the 55/45 mixture. The reaction mixture was then transferred to a 40 mL glass scintillation vial fitted with a screw cap incorporating a PTFE septum. For photoinitiated polymerization, the vial was placed on a set of steel rollers (VIVO electric 12 hotdog and 5 roller grill cooker; model hotdg-v005) and rotated at 3.25 rpm while being irradiated with an Everbeam 100W 365 nm UV LED Black light set positioned 9 cm above the steel rollers for 5 hours at room temperature. In the case of thermally-initiated polymerization, the vials were similarly rotated along their long axis (4-8 rpm) within an oven (UVP HB-1000 Hybridizer, or similar) set to 70° C. After polymerization, the reaction mixture was transferred to a 50 mL centrifuge tube, and the particles were isolated by centrifugation (4000 rpm, 3082g, 15 min). The particles were purified by three washes with 40 mL of acetone followed by one wash with 40 mL of ACN, accomplished by redispersing the particles in the solvent and then sedimentation by centrifugation. After purification, the particles were redispersed in 40 mL of ACN or DMF for storage or functionalization reactions. To measure isolated yields, an agitated 1 mL aliquot of the particle suspension in ACN was transferred to a pre-weighed 20 mL scintillation vial and dried under nitrogen for 1 h before transferring the vial into to a desiccator to dry in vacuo for four days at 20° C. A microscope image of the MED-60/0/10 particles (thermally-initiated) in DMF is shown in FIG. 6—Diameter: 1.74±0.22 μm (CV 0.13). Similar MED-57/0/10 particles made using a 57:43 ratio of MEK/heptane, a less polar solvent, had a diameter of 2.74±0.57 μm (CV 0.21).

MeAn can be an inefficient crosslinker because it has a strong propensity for cyclic polymerization, and because rearrangement of anhydride groups can lead to the loss of initially formed crosslinks. In addition, the conditions required for precipitation polymerization to microspheres (low total monomer loading, marginal solvency) tend to favor cyclic over acyclic propagation of MeAn. However, the presence of co-monomers, as in the present example where MeAn was paired with a permanent crosslinker, reduced the extent of MeAn cyclic polymerization and thus lead to more MeAn crosslinks. FIG. 7 shows a brightfield optical microscope image of MeAN-only (MED-55/0/0, photo) microspheres formed in 55/45 MEK/heptane in the absence of permanent crosslinkers. The MED-55/0/0 microspheres were formed in an isolated yield of 38% (Table 1), and remained intact when dispersed in MEK or DMF, solvents that would cause dissolution or merging of the particles if they were not crosslinked and consisted of linear pMeAn chains only. The successful formation of microspheres in reasonable yield demonstrated that MeAn acts as a crosslinker under these polymerization conditions.

Particles were obtained under the same polymerization conditions when the permanent crosslinkers EGDMA or DEGDMA, or mixtures of the two, were added at 10 mol % relative to total monomer. Particles prepared by photopolymerization in the presence of the permanent crosslinker(s) were obtained with about 40 to 55% yields of isolated particles (Table 1). The particle yields were higher, about 50 to 80%, for thermally-initiated polymerizations (Table 1).

TABLE 1
Isolated yields of methacrylic-based anhydride microspheres prepared by
precipitation polymerization

| Photoinitiation | | Thermoinitation | |
|---|---|---|---|
| Composition | Isolated Yield | Composition | Isolated Yield |
| MED-55/10/0 | 53% | MED-60/0/10 | 67 ± 10% (n = 7) |
| MED-55/5/5 | 48% | | |
| MED-55/3/7 | 44% | | |
| MED-55/2/8 | 42% | | |
| MED-55/2/8 | 38% | | |
| MED-55/0/10 | 54% | | |
| MED-55/0/0 | 38% | | |

The MEK/heptane mixed solvent selected for precipitation polymerization was advantageously a marginal solvent (with low viscosity) that does not react with the anhydride. An additional benefit of the MEK/heptane solvent system is that it allows fine-tuning of the solvency by varying the ratio of the two components.

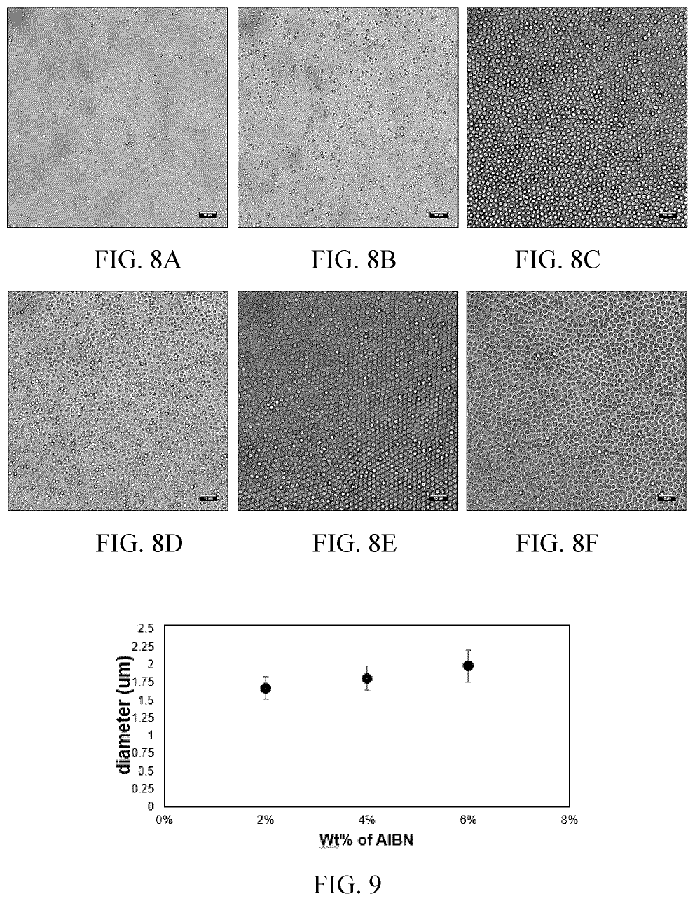

FIGS. 8A-8F show that narrow-disperse MED-55/5/5 (photo) particles can be made at monomer loadings up to 7%, and that the size increases with loading. Sizes in the range of no more than 1-3 μm were observed. Larger particles were seen with a 10% loading, where a size of 5-6 μm can be obtained, but the size dispersity was poor. The size of all particles will further increase after hydrolysis or functionalization.

FIG. 9 shows that the MED-62/0/10 (photo) particles increased in size gradually as the initiator concentration was increased and narrow-disperse particles were obtained. This was likely a result of higher monomer conversion.

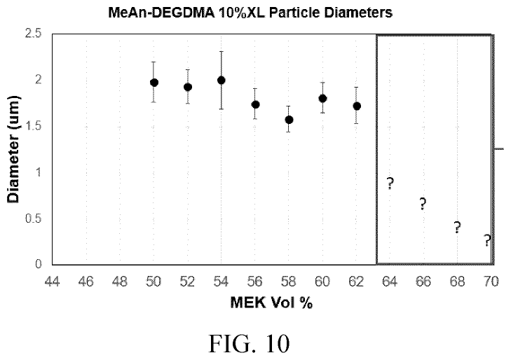

FIG. 10 shows the diameter of MED-X/0/10 (photo) particles made in MEK/heptane containing 50-70% MEK. Varying the solvent polarity, at least in this range, had little effect on the size. Particles with average diameters of about 2 μm and narrow-dispersity (CV≤0.1) were obtained for samples made in solvents with up to 62% MEK. The particles size will increase following hydrolysis or functionalization.

### Example 2

Anhydrides are hydrolyzed quite rapidly in aqueous media, which in the case of MeAn-based particles will lead to cleavage of the anhydrides (crosslinks, cyclic and pendant) and the creation of methacrylic acid or carboxylate groups depending on the pH. This will cause the particles to swell, especially at higher pH when the acid groups are deprotonated, or dissolve if there is no permanent crosslinker present. Anionic microgels were produced by hydrolysis of the MeAn-based microspheres. For example, purified MED-55/5/5 (photo) microspheres suspended in 40 mL of ACN were sedimented by centrifugation and resuspended in 5 mL of ACN before 11.3 mL of 1 M NaOH (1.1 eq.) was added. After 30 min, the mixture was diluted to 40 mL with distilled water, and then maintained at room temperature overnight under constant mixing by rotation at 20 rpm. The suspension of hydrolyzed microgels was transferred to cellulose dialysis tubing (3500 Da molecular weight cutoff (MWCO), Spectrum Laboratories) and were purified by dialysis against distilled water with daily water changes until the dialysate showed no absorbance by UV-Vis spectroscopy. The purified microgels were then freeze-dried to yield a white solid. To prepare sterile microgels, the freeze-dried microgels were soaked in 70% ethanol for 2 h, sedimented by centrifugation (3082 g, 15 mins), reswollen in sterile distilled-water, and then freeze-dried under sterile conditions using a Labconco aseptic adapter.

Microscope images of hydrolyzed MED-60/0/10 (thermal) particles are shown FIG. 6. At pH 7.4 (FIG. 6C) the particle diameter was 5.49□0.78 μm, and at pH 2 it was 1.6□0.4 μm, similar to the diameter of the particles before hydrolysis. Survival of the particles after hydrolysis showed that they were permanently crosslinked, and the dramatic swelling at pH 7.4 is consistent with the formation of a lightly crosslinked hydrogel. ¹H NMR analysis of a suspension of acidified (COOH-form) MED-60/0/10 (thermal) particles in DMSO-d6 showed signals at 0.7-2.2 ppm (CH₃, CH₂ backbone), 3.5-4.2 ppm (CH₂O) and 12.3 ppm (COOH) in a 76:8:12.5 ratio consistent with MAA:DEGDMA=93:7. This corresponds to MeAn:DEGDMA=87:13 since each MeAn molecule could give rise to two MAA units.

MED-55/0/0 (photo) particles, which lack a permanent crosslinker, dissolved when the anhydrides were hydrolyzed, while particles made with permanent crosslinkers such as MED-55/10/0 or -55/5/5 swelled and became more transparent but did not dissolve. Particles made with a greater fraction of DEGDMA such as MED-55/2/8 (photo) or MED-55/0/10 (photo) were very difficult or impossible to resolve by conventional optical microscopy after hydrolysis, reflecting extremely high degrees of solvation and swelling. An aqueous solution of MED-55/0/10 microgels were able to pass through a 0.45 μm pore syringe filter, but not a 0.22 μm syringe filter, suggesting that they are still particles though highly deformable.

The effect of pH on the swelling of MeAn-based anionic microgels with different permanent crosslinker compositions was determined by optical microscopy. Hydrolyzed MED-55/10/0, -55/5/5 and -55/0/10 made by photopolymerization were dispersed at 0.05 wt % in 100 mM phosphoric acid buffer (pH 2.4), 100 mM acetate buffer (pH 4.75), 100 mM phosphate buffer (pH 7.06), and 100 mM carbonate buffer (pH 10.0).

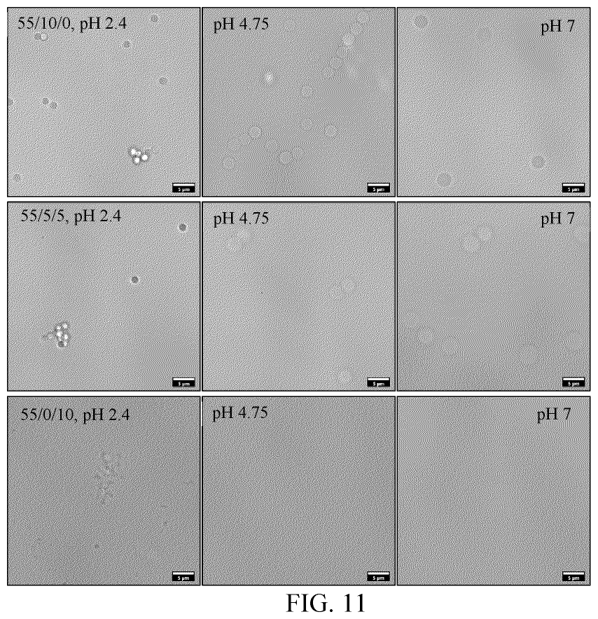

Swelling of the hydrolyzed particles provides information about the degree of crosslinking and about the stiffness of the hydrogels. Crosslinked poly(methacrylic acid) (pMAA) particles, such as those formed by hydrolysis of MED particles, collapse at low pH and are highly swollen at high pH when all of the MAA groups are ionized. Hydrolyzed MED particles made with 10 mol % permanent crosslinker but differing ratios of EGDMA and DEGDMA were suspended in solutions at pH 2.4, 4.75, and 7 (FIG. 11), where the carboxylic acid groups should be fully protonated (neutral), half ionized and fully ionized, respectively. The particles clearly swell as the pH was increased, and became more difficult to resolve as their refractive index became closer to that of the solution.

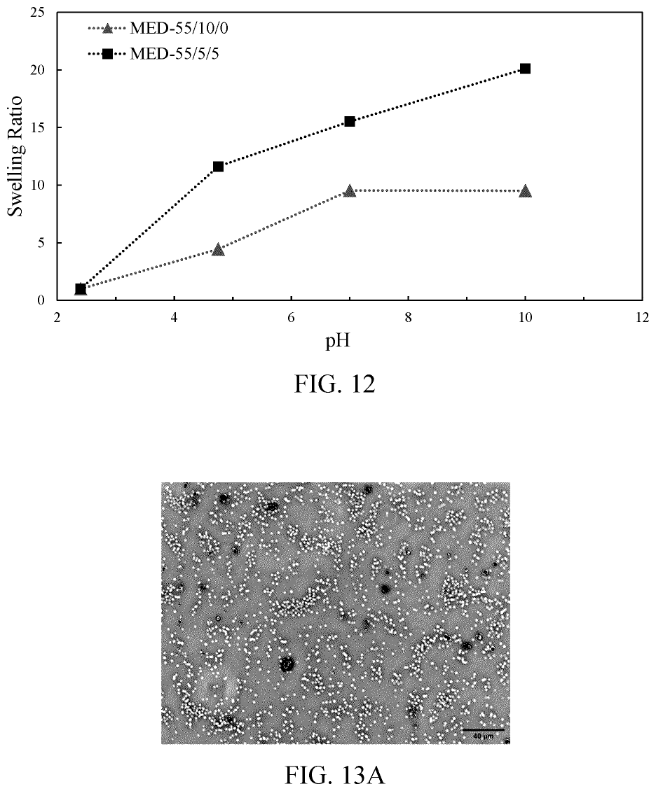

The degree of swelling was estimated by comparing the volume of the collapsed particle at pH 2.4 with the volume at higher pH using the relationship (Dₓ/D_{2.4})³, where Dₓ is the particle diameter at a given pH and D_{2.4} is the diameter at pH 2.4. As shown in FIG. 12, the particles underwent considerable swelling (≥10×) at pH 7 showing that they were lightly crosslinked hydrogels. The swelling was increased significantly by increasing the DEGDMA content in the permanent crosslinker. Greater swelling is consistent with a crosslinker that is longer and more hydrophilic than EGDMA but the magnitude of change was unexpected. The results demonstrate that using crosslinkers with longer spacers such as oligo/poly (ethylene glycol) methacrylate, and hydrophobic alkyl spacers such as butanediol dimethacrylate, can be used to further tune the size and swellability of the microgels.

### Example 3

The polymer-bound anhydride groups can also be used for post-polymerization modification via reaction with nucleophiles like amines, alcohols or thiols, leading to incorporation of the modifier, formation of polymer-bound carboxylic acid groups, and cleavage of anhydride crosslinks. Particles with a broad range of properties can be obtained by reaction of MeAn-based particles with one or more of the wide variety of modifiers available. It is possible to vary the hydrophobicity and charge of the particles, and to introduce groups that provide a variety of useful properties (e.g., fluorescent or radiolabels, cell-binding, drug release, etc.). Functionalization of MeAn-based particles enables the preparation of particles that would be otherwise inaccessible by direct precipitation polymerization of the structurally analogous monomer units, and depending on the modifier and conditions chosen, it is also possible to control whether modification happens throughout the particle or is largely restricted to the particle surface. To demonstrate functionalization, particles such as those made in Example 1 were reacted with reagents that were fluorescent, cationic and/or cell-binding motifs as described below.

### Polyampholyte Microciels

MeAn-containing particles were functionalized with an excess of DMAPA to make polyampholyte microgels. For example, 50:50 cationic:anionic polyampholyte microgels were targeted by adding DMAPA (3.17 g, 31.1 mmol) to MED-55/5/5 (photo) microspheres suspended in 40 mL of ACN in a 50 mL centrifuge tube. This corresponds to a roughly sixfold excess of DMAPA relative to the amount of MeAn in the particles. The reaction was maintained at room temperature overnight while being mixed by rotation of the vial/tube at 20 rpm. After reaction, the microgels were sedimented by centrifugation (3082 g, 15 min) and then washed once by resuspending in 40 mL ACN followed by centrifugation. After washing, the microgels were dispersed in 40 mL distilled water and allowed to swell before being transferred to cellulose dialysis tubing (3500 Da MWCO). The microgels were first dialyzed twice against 0.9 wt/v % NaCl over 2 days, followed by dialysis against distilled water changed daily for 4 days. The microgels were then freeze-dried to give a white solid that was then sterilized as described above.

### Fluorescently-Labeled Microgels

As-formed MeAn-based microspheres were fluorescently labeled with TAMRA

targeting a degree of labeling of 0.025-0.05 mol % relative to MeAn units. TAMRA-cadaverine (222 μL of a 0.2 wt % solution in DMF; 0.86 μmol) was added to a suspension of MeAn-based microspheres in 30 mL of ACN that contained about 0.53 g (3.45 mmol) of polymeric MeAn groups, and then mixed for 2 days at room temperature (22° C.). The particles were isolated by centrifugation followed by three washes with 40 mL ACN. The microspheres were then resuspended in 40 mL of distilled water, mixed for 1 day at room temperature (22° C.), and then isolated by centrifugation before being resuspended in 10 mL of distilled water, and dialyzed in water using 3500 Da MWCO cellulose acetate tubing. The water bath was changed daily until the absorbance measurements of the dialysate reached 0, indicating no further elution of small molecules. The particles were isolated, resuspended in 40 mL of 70% (v/v) ethanol for 1 h for sterilization. The ethanol suspension was centrifuged, and then transferred to a biosafety cabinet where the supernatant was removed. The particles were dispersed in 30 mL of sterile water, frozen in dry ice and lyophilized with an aseptic adapter (Labconco) to provide TAMRA-labelled particles as a pink solid.

### RGD-Functionalized Microgels

MeAn-based microspheres were functionalized with both TAMRA and RGD with targeted degrees of functionalization (w.r.t. MeAn) of about 0.025 and 0.5 mol %, respectively. A solution of 5.3 mg (15.3 μmol) RGD in 2 mL of 1:1 (v/v) ACN/DMF was added to a 30 mL suspension of MeAn-based microspheres in ACN, containing approximately 0.53 g (3.45 mmol) of MeAn units, and then approximately 10 min later, TAMRA-cadaverine (222 μL of a 0.2 wt % solution in DMF; 0.86 μmol) was added. The reaction, washing and isolation steps were conducted as described previously in the present example. The freeze-dried RGD- and TAMRA-functionalized microspheres were isolated as a pink solid.

### Properties of Functionalized Particles

¹H NMR analysis of the DMAPA-functionalized MED-57/0/10 (thermal) particles in D₂O showed signals at 0.7-2.4 ppm (CH₃, CH₂ backbone), 2.85 ppm (N(CH₃)₂), 3.15 ppm (N-CH₂), and 3.5-4.2 ppm (CH₂O) in a 15.8:6:3.7:1.5 ratio consistent with MAA:DMAPMA (acid/amine)=58:42. Particles resuspended in PBS at pH 2 and pH 7.4 were examined by microscopy. The particle diameter was 6.57±1.01 μm at pH 7.4 and 7.06±1.61 μm at pH 2. The large diameters cf. the precursor particle (Diameter in DMF: 2.74±0.57 μm; CV 0.21) shows that the temporary crosslinks have been cleaved and a lightly crosslinked hydrogel produced. In contrast to the hydrolyzed particles in Example 2, the particles do not collapse at low pH. This shows that the functionalization was successful as the presence of charged groups in the form of ammonium ions ensures that the particles remain swollen at low pH.

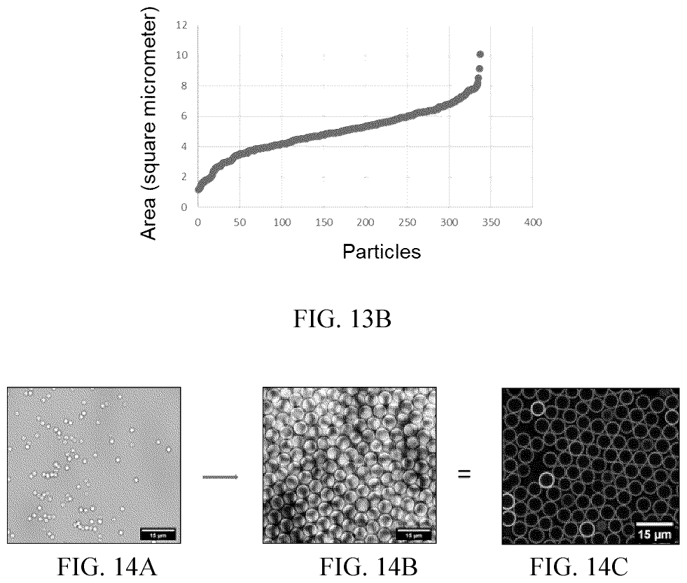

A microscope image of MED-60/0/10 (thermal) particles that had been functionalized with N,N-dimethylethylenediamine (DMEDA) and then dispersed in HEPES-buffered saline (pH 7.6) is shown in FIG. 13A. In FIG. 13B is a plot of particle area showing that most of the particles have areas between 4 and 6 μm², which corresponds to particle diameters of 2.25 to 2.75 μm.

Images of MED-55/5/5 (photo) particles before and after functionalization with DMAPA and TAMRA-cadaverine (0.05 mol %) are shown in FIGS. 14A-C. The particles underwent dramatic swelling upon functionalization and dispersal in aqueous solution, and were able to form a close-packed array because they are of fairly uniform size (FIG. 14B). Confocal fluorescence microscopy revealed that the TAMRA label was concentrated at the particle surface, perhaps because the TAMRA-cadaverine, which was added before DMAPA, reacted with the first MeAn groups that were encountered. This demonstrated the ease of particle functionalization, and the ability to localize different modifiers depending on the order of addition and/or molecular weight.

Reaction of the MeAn groups in the particles with di- or polyamines allows the particle charge to be tailored for specific biomaterial applications starting from the same base scaffold particles. While hydrolysis of MeAn yields particles that are anionic at neutral pH, complete reaction of the anhydride groups with a diamine such as DMAPA would yield a polyampholyte particle with an approximately 1:1 charge ratio. MED-55/0/10 (photo) particles functionalized with an excess of DMAPA were suspended in D₂O and analyzed by ¹H NMR. They were found to have an approximately 80% degree of functionalization, corresponding to a cationic:anionic charge ratio of 40:60, close to the targeted 50:50 ratio. The slight excess anionic charge may be a result of incomplete functionalization, the presence of some MAA in the MeAn starting material (94% purity), and/or inadvertent hydrolysis of some anhydride groups prior to functionalization.

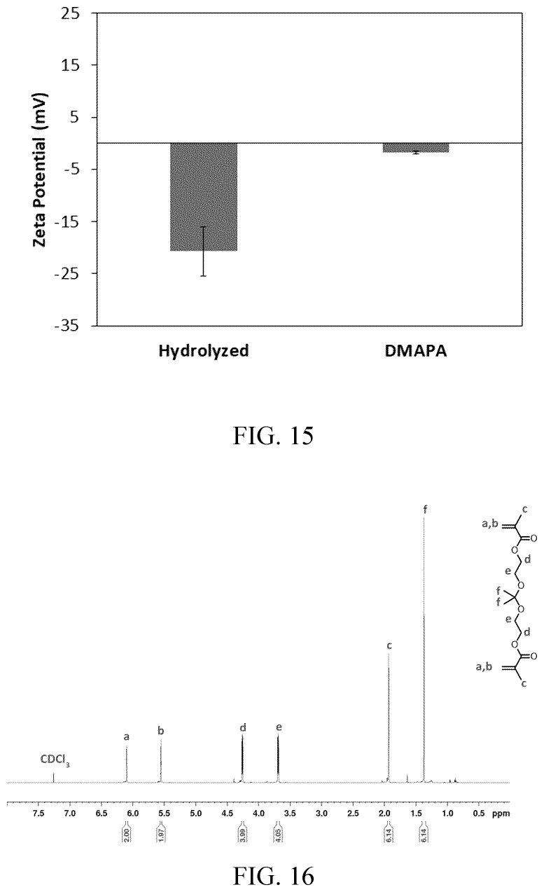

MED-55/10/0 (photo) microspheres that had been only hydrolyzed, or functionalized with DMAPA, were dispersed in PBS at pH 7.4 at a concentration of 0.25 wt %. The zeta potential of each particle sample was measured using a Zetasizer Nano ZS from Malvern. Approx. 700 μL of the particle dispersion was transferred to a Malvern Zeta-Dip Cell and measurements (n=3) were performed at 25° C. Zeta potential measurements, which reveal the charge on the particle surface, were carried out for the hydrolyzed and DMAPA-functionalized MED-55/10/0 particles (FIG. 15). Hydrolyzed MED-55/10/0 particles showed a strongly negative zeta potential (−20.7±4.7 mV) at physiological pH, as expected for lightly crosslinked pMAA particles. The DMAPA-functionalized particles had a zeta potential close to zero (−1.82±0.27 mV), consistent with a polyampholyte with a −1:1 charge ratio.

### Example 4

In some biomaterial applications it is desirable to incorporate controlled degradation of materials under physiological conditions. Acid-labile functional groups, such as acetals and ketals, are of interest due to their degradation under acidic conditions while maintaining stability at basic conditions. To demonstrate the particle degradation, a ketal-containing crosslinker, (propane-2,2-diylbis(oxy))bis(ethane-2,1-diyl) bis(2-methylacrylate) (called KTMA here) was prepared by the acid-catalyzed reaction of 2-hydroxyethyl methacrylate with dimethoxypropane. KTMA is structurally similar to DEGDMA and it was expected that it might be used under the same polymerization conditions.

KTMA was synthesized using a procedure based on previously reported syntheses.^{12,13} HEMA (10.0 g, 76.8 mmol), DMPA (3.805 g, 36.5 mmol), pTSA (0.157 g, 0.825 mmol), and MEHQ (0.20 g, 0.2% w.r.t. HEMA) were charged to a 25 mL pear-shaped flask equipped with a magnetic stir bar. The reaction mixture was heated at 60° C. overnight in an oil bath with nitrogen gas bubbling to remove methanol. The resulting dark red reaction mixture was cooled to room temperature, and then passed through a 100 g silica plug using an 80/20 hexanes/ethyl acetate eluent. The product-containing fractions were combined and concentrated in vacuo to give KTMA as a pale-yellow liquid in 21% yield and approx. 95% purity. The ¹H NMR (CDCl₃, 600 MHz) of KTMA is shown in FIG. 16: δ6.09 (2H, s), 5.55 (2H, s), 4.26 (4H, t), 3.69 (4H, t) 1.93 (6H, s), 1.37 (6H, s).

KTMA-crosslinked MeAn microparticles were synthesized using the procedure described above (Example 1), but with the degradable KTMA crosslinker in place of EGDMA and/or DEGDMA. For example, MKT-55/15 particles (15 mol % KTMA, 55/45 MEK/heptane) were made by photoinitiated polymerization using MeAn (1.284 g, 8.3 mmol), KTMA (0.448 g, 1.49 mmol), and AIBN (0.036 g, 0.022 mmol) dissolved in 35.15 mL of a 55/45 MEK/heptane. After isolation and washing, the supernatant was removed, and NaOH solution (20 mol excess relative to MeAn units) was added to hydrolyze the anhydride groups. The reaction mixture was left for 1 day, before the particles were isolated by centrifugation, and resuspended in 40 mL of distilled water. The suspension had pH ˜11, which is important to prevent premature degradation of the ketal groups.

Indeed, narrow-disperse, KTMA-crosslinked, spherical particles were obtained under the same conditions employed with EGDMA and DEGDMA as evidenced by the formation of close-packed particle arrays (FIG. 17). After hydrolysis, particles made with 10 mol % KTMA (MKT-55/10) were highly swollen and degraded fairly quickly at physiological pH. When the KTMA loading was raised to 15 mol % (MKT-55/15), more long-lived particles were obtained.

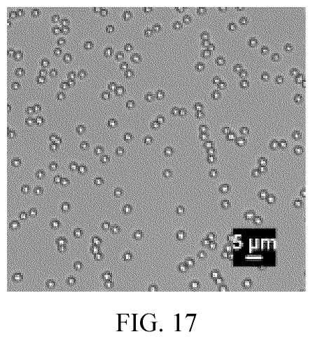

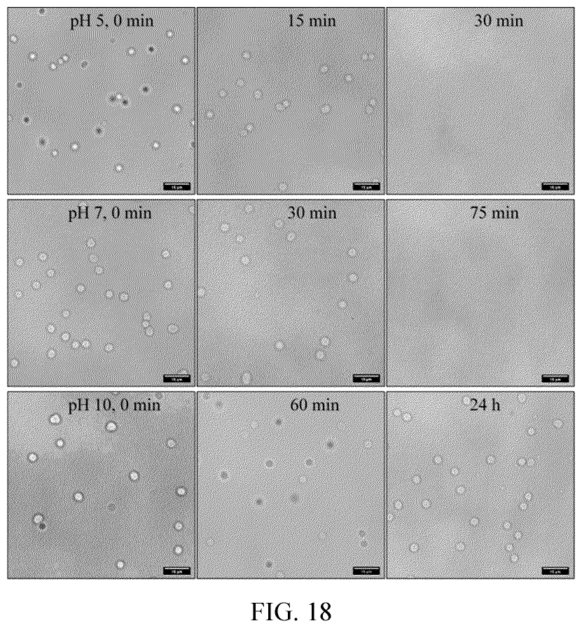

Anionic MKT-55/15 microgels, prepared by selective hydrolysis of the anhydride groups under basic conditions, were dispersed in buffers at pH 5, 7 and 10 at room temperature to probe the rate of particle degradation (FIG. 18). At pH 5, the particles visibly swell within 15 min and have disappeared after 30 min, while at pH 7 it takes 75 min. The particles remain intact after 24 h at pH 10. The accelerated rate of degradation at low pH is in line with the acid catalyzed mechanism of hydrolysis for ketals and acetals. Slower degradation can be achieved by using higher KTMA loading, introducing hydrophobicity via functionalization or copolymerization, or by changing the nature of the ketal linkage.

### Example 5

The following example shows that the residual vinyl groups from the permanent crosslinker may be used to graft a polycation onto the hydrogel microparticles.

The hydrolyzed particles of Example 2 were grafted with 2-(N,N-dimethylamino)ethyl acrylate (DMAEA). Hydrolyzed MeAn-DEGDMA (90:10) particles in the acid (COOH) form (0.100 g) were combined with 10 mL DMF, 1.00 g DMAEA (7.00 mmol), 11.5 mg (1 mol %) AIBN, and optionally 28.0 mg (1 mol %) fluorescein O-methacrylate. The solution was bubbled with nitrogen for 45 min and then heated for 18 h in an oil bath at 70° C. ¹H NMR (DMSO-d6) of an aliquot of the reaction mixture indicated that there had been 58% monomer conversion for the reaction with fluorescein O-methacrylate and 83% for the one without. The particles settled once the reaction mixture was cooled, and the yellow supernatant was removed. The particles were washed once by dispersing in acetone (˜45 mL) followed by centrifugation, and then they were dispersed in 11 mL of ˜0.1 M HCl. The suspension was adjusted to pH 2.2 using 0.1 M NaOH before it was transferred to dialysis tubing (1 MDa molecular weight cutoff) and dialyzed in 1 mM HCl (4 L) for 2 days with one change of the bath. The particles were isolated by freeze-drying. The ¹H NMR spectrum of a suspension of the particles in DMSO-d6 included signals at 0.7-2.2 ppm (CH₃, CH₂, CH backbone), 2.8 ppm (N(CH₃)₂), and 12.3 ppm (COOH) in a 26.7:6:3.89 ratio consistent with MAA:DMAEA=80:20.

### Example 6: Cryopreservation

This example shows that the hydrogel microparticles as prepared in the Examples above can act as cryoprotective agents.

NIH 3T3 murine fibroblasts cells were cultured in T-75 tissue culture-treated flasks with DMEM supplemented with; 10% v/v BCS, 1% v/v penicillin-streptomycin, and the cells were maintained in a 37° C., 5% CO₂ incubator. When the cells reached 70-90% confluency they were washed with PBS and incubated at 37° C. for 2 minutes with a 0.05% trypsin-EDTA solution in PBS to detach the cells. The trypsin-EDTA was quenched by addition of supplemented DMEM, and the cells were collected and transferred to a 15 mL centrifuge tube. The cells were spun down by centrifugation at 300 g for 5 mins and resuspended in 5 mL of supplemented DMEM. A 50 μL aliquot of the resuspended cells was stained with 50 μL of 0.4% trypan blue and the cell viability and concentration were measured with an Invitrogen Countess automated cell counter. The cells were prepared for cryopreservation by transferring aliquots of the resuspended cells to 15 mL centrifuge tubes using an appropriate volume of cell suspension to achieve 4 million cells per tube. The cells were spun down at 300 g for 5 mins and resuspended in 1 mL of cryoprotective solution, to achieve a cell concentration of 4 million cells/mL, and then transferred to 2 mL polypropylene cryotubes. The cryotubes were placed in a Mr. Frosty container filled with iso-propanol, and the container was then placed in a −80° C. freezer, resulting in a cooling rate of approximately 1° C./min. After 24 h, the frozen samples were thawed in a 37° C. water bath for 2 min, after which they were diluted into 9 mL of pre-warmed (37° C.) DMEM and sedimented at 300 g for 5 min. The cells were resuspended in 1 mL of DMEM, and then 50 μL aliquots of each sample were stained with 50 μL of 0.4% trypan blue and the cell viability and concentration were measured. To monitor cell attachment and growth after thawing, the remainder of each cryopreservation sample was divided into three 300 μL aliquots which were seeded into three wells of a 6-well tissue culture treated plate where each well contained 3 mL of DMEM. The plates were maintained in a 37° C., 5% CO₂ incubator and monitored for 7 days after thawing. Any samples that reached confluency during this time were transferred from the 6-well plates to T-75 flasks by detachment with trypsin-EDTA. On days 3, 5 and 7 after thawing, the cells in one well for each sample were washed with PBS and detached by incubation with 0.025% trypsin-EDTA for 2 mins at 37° C. The detached cells were collected and transferred to 15 mL, spun down at 300 g for 5 mins, and resuspended in 1 mL of DMEM before 50 μL aliquots were stained with 50 μL of 0.4% trypan blue and counted with a Countess automated cell counter.

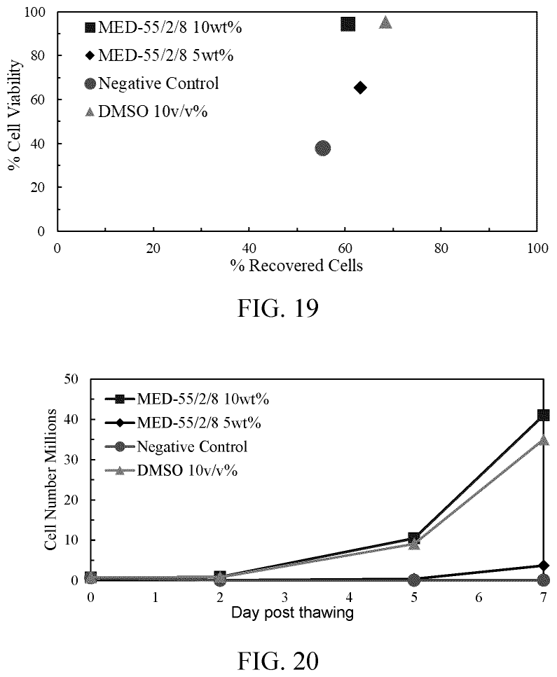

A 24-hour freeze/thaw cycle using a standard mammalian cell freezing procedure was used to freeze NIH 3T3 cells with MED-55/2/8 (photo) DMAPA-functionalized polyampholyte microgels at 10 and 5 wt/v % concentration in DMEM alongside a positive control of 10% v/v DMSO and negative control of just DMEM. FIG. 19 shows the immediate post-thaw viability and percentage of cells recovered as measured by trypan blue staining.

Cells frozen with 10 wt/v % microgels showed post-thaw viability and recovery comparable to that of the 10% v/v DMSO sample, with the DMSO sample having a slightly higher % recovery. Both high cell viability and % recovery are important measures of the effectiveness of cryoprotective agents. For linear polyampholytes, cell death from ice-crystal formation leading to cell fragmentation can give an over-estimation of the effectiveness as a cryoprotective agent. The fragmented dead cells are not detected leading to a higher measured cell viability but lower % recovery.¹⁴ FIG. 19 also shows an increased effectiveness going from 5 to 10 wt/v % of the microgels, this trend is in line with previous reports on linear polyampholytes for cryopreservation and may be a result of improved cellular dehydration during freezing, which prevents intracellular ice crystal formation, decreased ice crystal size, and reduced cell sedimentation during freezing.^{14,15,16}

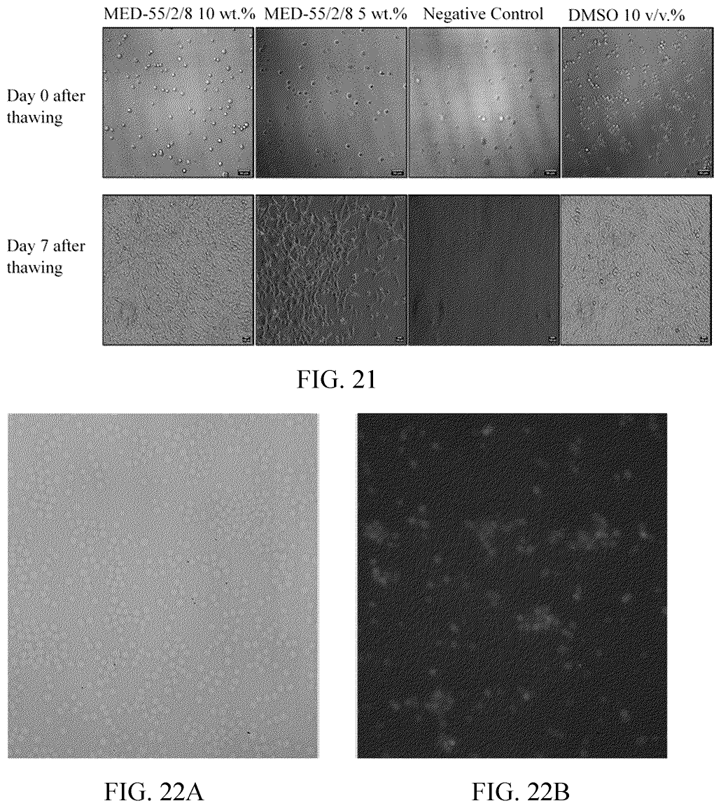

In addition to immediate post-thaw measures, thawed cells were seeded onto tissue culture plates to observe cell attachment and growth as longer-term measures of cellular health. As shown in FIG. 20, cell attachment and growth of the sample frozen with 10 wt/v % microgels was similar to that of cells frozen in 10% v/v DMSO. In addition, brightfield images of attached cells show normal healthy 3T3 morphology after attachment (FIG. 21). Interestingly, by this measure the polyampholyte microgels seem to demonstrate improved effectiveness as cryoprotective agents compared to analogous linear polyampholytes. Although the mechanism of cryopreservation with linear polyampholytes is not clear, a key step is thought to be conformal coating of the polymer around the cells during freezing,¹⁷ and it is possible that the incomplete removal of this coating from the cells after thawing may lead to the observed poor attachment and proliferation. Without wishing to be bound by a theory, the soft polyampholyte microgels, which can deform around the cells to provide a conformal coating, but are then more easily separated from the cells by differential sedimentation, may lead to better cell attachment and growth.

### Example 7: Protein (Antigen) Binding

The following example shows that cationic microparticles such as those prepared in Example 5 can bind ovalbumin, an antigen.

4 mL of a 1 mg/mL solution of pDMAEA-grafted microparticles (prepared as described in Example 5) in distilled water was added to approx. 100 μL of a 1 wt. % solution of fluorescein-labelled ovalbumin (OVA-FITC) in phosphate-buffered saline (PBS) at pH 7.4. The solution was mixed vigorously for about 1 min using a vortex mixer, and then centrifuged for 1 min at 4000 rpm to isolate the particles. The particles were resuspended in 1 mL of PBS before being examined by fluorescence microscopy. As shown in FIGS. 22A and 22B, the particles become fluorescent revealing that they have bound OVA-FITC.

### Example 8: Microgel Cellular Uptake

This example shows the cellular uptake of appropriately functionalized microgels, in this case with RGD, a cell-attachment motif, as prepared in Example 3.

NIH 3T3 murine fibroblasts cells were cultured to 70-90% confluency, detached and counted. After counting, the cells were sedimented at 300 g for 5 mins and then resuspended in the appropriate volume of DMEM to achieve a cell concentration of 2.0×10⁶ cells/mL. To form microgel-cell composite clusters, three stock solutions (2 wt/v % in DMEM) were prepared from MED-55/5/5 (photo) microgels that were: a) TAMRA-functionalized, b) TAMRA- and RGD-functionalized, and c) TAMRA- and DMAPA-functionalized. In the wells of a Cellvis 96-well glass bottom plate, a series of 200 μL/well samples containing 1.0×10⁶ cells/mL and varying concentrations (0.01 to 1.0 wt/v %) of one of the three microgels compositions were prepared by combining aliquots of the cell suspension, microgel stock solutions, and DMEM. The cells were incubated with the microgels for 3 days at 37° C. to allow for cell/microgel interaction prior to imaging. After the 3-day incubation, the cells were stained with 50 μL of a 10 μmol solution of Calcein-AM in PBS and imaged on a Nikon Al Confocal Ti Eclipse microscope.

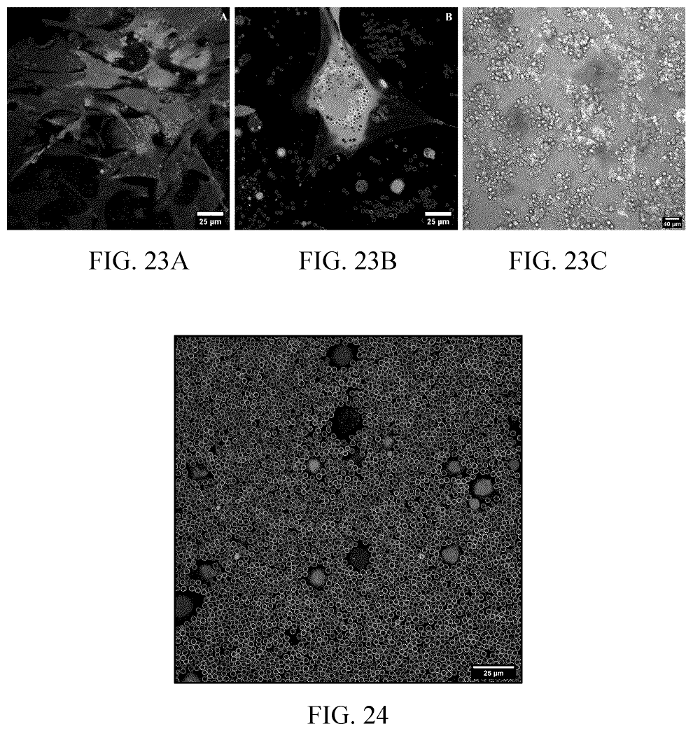

Particle functionalization can be used to incorporate modifiers that promote cell binding or internalization. MED-55/5/5 (photo) particles were modified with RGD to promote cell-binding and TAMRA to facilitate visualization of the particles (Example 3). NIH 3T3 cells were co-cultured for 3-days with three different types of TAMRA-labeled MED-55/5/5 particles: A) DMAPA-functionalized polyampholyte, B) RGD-functionalized anionic, and C) anionic. Following incubation, the cells were stained with calcein-AM and imaged by confocal microscopy (FIG. 23A-C). Cells incubated in the presence of microgels functionalized with RGD, a tripeptide attachment motif, were found to be internalized by the cells as seen in FIG. 23B with the appearance of TAMRA-labeled microgels (red) within the cells. It also appears that the particles exclude the Calcein stain, which results in the appearance of holes within the cells when viewed through the FITC channel. Confocal images of the 3T3 cells incubated with the DMAPA-functionalized polyampholyte particles (FIG. 23A), or anionic particles (FIG. 23C) show no evidence of internalization. This suggests that the incorporation of RGD on the microgels promoted cellular uptake of the microgels and had the potential to be used for delivery of payloads to cells such as antigens for vaccine drug delivery.

### Example 9: Cell Scaffolds and Co-Encapsulation of Cells and Microgels

DMAPA functionalized polyampholyte MED-55/15/0 (photo) microgels fluorescently labeled with 0.05% TAMRA cadaverine relative to MeAn-groups were prepared as described in Example 3 and after hydrolysis were purified by centrifugation/resuspension three times with distilled water and twice with PBS and then resuspended in 40 mL of PBS. NIH 3T3 murine fibroblasts cells were cultured to 70-90% confluency in a T75 cell culture flask, detached and counted as described above, and resuspended to an approximate cell concentration of 6.0×10⁶ cells/mL in PBS. To the resuspended cells 34.7 μL of a 1 mg/mL calcein AM solution was added to stain the cells at a concentration of 4 μM calcein AM. In a 96-well glass bottom plate; 50 μL of stained cells, 50 μL of fluorescently labeled MED-55/15/0 (photo) anionic microgels and 200 μL of PBS were mixed and imaged on a Nikon Al Confocal Ti Eclipse microscope.

There is interest in using hydrogel particles as cell scaffolds because they can form a 2- or 3-D array and the properties of the scaffold can be fine-tuned by mixing particles with differing size, stiffness or chemistry. The facile functionalization of MeAn-based particles makes them ideal starting materials for the manufacture of components in a particle scaffold. FIG. 24 shows a simple demonstration of a 3-D particle scaffold with NIH 3T3 cells (stained with Calcein-AM, green) dispersed amongst TAMRA-labeled polyampholyte MED-55/15/0 (photo) microgels.

The ease of handling particles means that they may be easily combined with cells in a more confined geometry such as a capsule. Calcium alginate is often used to encapsulate cells but sometimes provides a less than ideal environment for cells in terms of viability, differentiation, or proliferation. Co-encapsulation of cells with particles bearing suitable binding or signaling motifs may provide an improved environment. As a demonstration, NIH 3T3 cells were co-encapsulated with varying concentrations (0.001 to 0.5%) of MED-55/10/0 (photo) polyampholyte microgels in calcium alginate capsules. The capsules were subsequently given a protective polycation/polyanion coating before Live/Dead staining of the encapsulated cells with calcein-AM and ethidium-homodimer.

NIH 3T3 murine fibroblasts cells were co-encapsulated with MED-55/10/0 (photo) anionic microgels in calcium-alginate capsules. A 4×10⁶ cells/mL solution of 3T3 cells were prepared in a pH 7.4 35 mM HEPES-buffered saline solution and combined in various ratios with: 2 wt/v % solution of Na-Alginate in pH 7.4 35 mM HEPES-buffered saline, 2 wt/v % solution of MED-55/10/0 anionic microgels in pH 7.4 35 mM HEPES-buffered saline, and pH 7.4 35 mM HEPES-buffered saline to achieve solutions at a total volume of 1 mL and concentrations of 0.5, 0.05, and 0.001 wt/v % of the microgels at a constant cell and Na-alginate concentration of 2×10⁶ cells/mL and 1.0 wt/v % Na-alginate respectively. The prepared solutions were loaded into three 1 mL BD plastic syringes and the capsules were formed by extrusion through a ramé-hart 20G coaxial needle at a solution flowrate of 15 mL/hr controlled by a Harvard Apparatus syringe pump and a coaxial air flow of 2.25 L/min to shear off small droplets into a 100 mM CaCl₂, 45 mM NaCl, and 35 mM HEPES pH 7.6 gelling bath. The formed capsules were then allowed to gel for 5 minutes in the gelling bath after completion of extrusion. The formed capsules were collected and washed, and then coated with poly-L-lysine (PLL) and partially (50%) hydrolyzed poly(methyl vinyl ether-alt-maleic anhydride) (PM50) to form capsules with covalently crosslinked shells. The coated capsules were transferred to 60 mm petri dishes containing 5 mL of DMEM supplemented with 10% v/v BCS and 1 v/v% penicillin-streptomycin and maintained in a 37° C., 5% CO₂ incubator. After 1-day of incubation, approximately 200 μL aliquots of capsules prepared at the three concentrations were transferred to a 96-well glass bottom plate and stained for 30 minutes with 50 μL of a 10 μm and 50 μL of 4 μm ethidium homodimer solutions prepared in 35 mM HEPES-buffered saline prior to imaging on a Nikon A1 Confocal Ti Eclipse microscope.

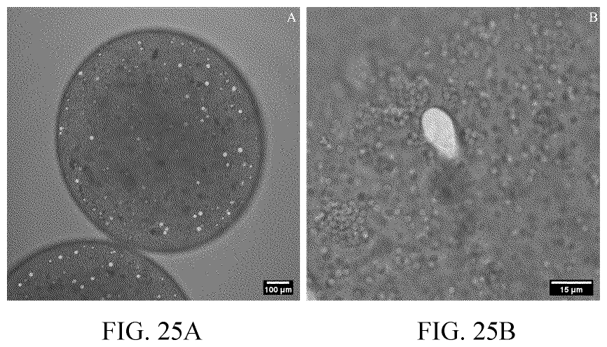

As shown in FIGS. 25A-B, the particles did not interfere with the encapsulation process, and the encapsulated cells showed high viability in the presence of the particles. The capsule shown in FIG. 25A had the highest particle loading, which impacted the transparency of the capsules. FIG. 25B shows a 3T3 cell in close contact with the particles within the capsule. While the cells might attach and spread on these polyampholyte particles, attachment and spreading would be promoted by including cell binding motifs allowing the particles to serve as granular cell attachment points.

### References

¹Caldwell, A. S., Campbell, G. T., Shekiro, K. M. T., & Anseth, K. S. (2017). Clickable Microgel Scaffolds as Platforms for 3D Cell Encapsulation. Advanced Healthcare Materials, 6(15), 1700254-8. http://doi.org/10.1002/adhm.201700254
²X. Yin, H. D. H. Stöver, Macromolecules 2003, 36, 8779-8779.
³X. Yin, H. D. H. Stöver, Macromolecules 2003, 36, 9817-9822.
⁴Li, W., Lee, S., Ma, M., Kim, S. M., Guye, P., Pancoast, J. R., et al. (2013). Microbead-based biomimetic synthetic neighbors enhance survival and function of rat pancreatic β-cells. Scientific Reports, 3. http://doi.org/10.1038/srep02863
⁵R. Takekoh, W. Li, N. A. D. Burke and H. D. H. Stöver, Journal of the American Chemical Society. 2006. 128(1) 240-244.
⁶A. Dubey, N. A. D. Burke, H. D. H. Stöver, J. Polym. Sci., Part A: Polym. Chem. 2015, 53, 353-365.
⁷R. M. Kleinberger, N. A. D. Burke, C. Zhou, H. D. H. Stöver, J. Biomater. Sci, Polym. Ed. 2016, 27, 351-369.
⁸G. B. Butler, Acc. Chem. Res. 1982, 15, 11, 370-378.
⁹Briard, J. G., Poisson, J. S., Turner, T. R., Capicciotti, C. J., Acker, J. P., & Ben, R. N. (2016). Small molecule ice recrystallization inhibitors mitigate red blood cell lysis during freezing, transient warming and thawing. Scientific Reports, 6, 23619. http://doi.org/10.1038/srep23619
¹⁰Tsuboyama K, Osaki T, Matsuura-Suzuki E, Kozuka-Hata H, Okada Y, Oyama M, et al. (2020) A widespread family of heat-resistant obscure (Hero) proteins protect against protein instability and aggregation. PLoS Biol 18(3):e3000632. https://doi.org/10.1371/journal.pbio.3000632.
¹¹A Keith Dunker, Israel Silman, Vladimir N Uversky, Joel L Sussman. Function and structure of inherently disordered proteins. Current Opinion in Structural Biology 2008, 18, 756-764.
¹²Syrett, J. A.; Haddleton, D. M.; Whittaker, M. R.; Davis, T. P.; Boyer, C. Functional, Star Polymeric Molecular Carriers, Built from Biodegradable Microgel/Nanogel Cores. Chem. Commun. 2011, 47 (5), 1449-1451. https://doi.org/10.1039/c0cc04532b.
¹³Bhuchar, N.; Sunasee, R.; Ishihara, K.; Thundat, T.; Narain, R. Degradable Thermoresponsive Nanogels for Protein Encapsulation and Controlled Release. Bioconjug. Chem. 2012, 23 (1), 75-83. https://doi.org/10.1021/bc2003814.
¹⁴Zhao, J.; Johnson, M. A.; Fisher, R.; Burke, N. A. D.; Stöver, H. D. H. Synthetic Polyampholytes as Macromolecular Cryoprotective Agents. Langmuir 2019, 35 (5), 1807-1817. https://doi.org/10.1021/acs.langmuir.8b01602.
¹⁵Matsumura, K.; Hyon, S. H. Polyampholytes as Low Toxic Efficient Cryoprotective Agents with Antifreeze Protein Properties. Biomaterials 2009, 30 (27), 4842-4849. https://doi.org/10.1016/j.biomaterials.2009.05.025.
¹⁶Matsumura, K.; Bae, J. Y.; Hyon, S. H. Polyampholytes as Cryoprotective Agents for Mammalian Cell Cryopreservation. Cell Transplant. 2010, 19, 691-699. https://doi.org/10.3727/096368910X508780.
¹⁷Rajan, R.; Hayashi, F.; Nagashima, T.; Matsumura, K. Toward a Molecular Understanding of the Mechanism of Cryopreservation by Polyampholytes: Cell Membrane Interactions and Hydrophobicity. Biomacromolecules 2016, 17 (5), 1882-1893. https://doi.org/10.1021/acs.biomac.6b00343

1. A method for producing microparticles comprising:
combining at least one temporary crosslinker and at least one permanent crosslinker in an organic solvent having a polarity suitable for a precipitation polymerization to occur; and
allowing the precipitation polymerization to take place thereby forming the microparticles having polymers comprising monomers of the temporary crosslinkers and the permanent crosslinkers.

2. The method according to claim 1, wherein a total monomer loading before the precipitation polymerization is calculated as the combined loading of the at least one temporary crosslinker, the at least one permanent crosslinker, and any other monomers, and has a value of between 1 to 20 weight %.

3. The method according to claim 1, wherein a total crosslinker loading before the precipitation polymerization is the combined loading of temporary crosslinker and permanent crosslinker and has a value of more than 10 mol %, and wherein the ratio of temporary crosslinker to permanent cross linker is between 50:50 and 99:1 mol %.

4. The method according to claim 1, wherein the solvent is 4 to 5 MPa^{1/2} above or below that of the polymers.

5. The method according to claim 1, wherein the solvent is selected from the group consisting of acetonitrile, methyl ethyl ketone, heptane, and combinationsof methyl ethyl ketone and heptane thereof.

6. The method according to claim 1, wherein the temporary crosslinker is of formula (I) or (Ila)-(Ilf) 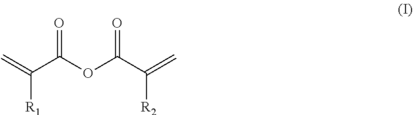
wherein R₁ and R₂ are independently selected from H, C₁-C₄ linear or branched carbon chain, benzyl, phenyl or OJ, where J is defined as a C₁-C₄ linear or branched carbon chain; 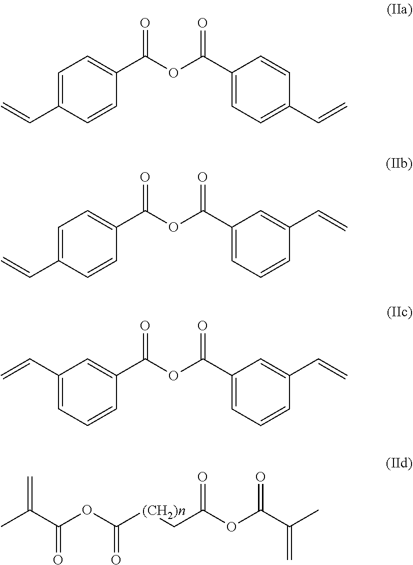
and wherein n is an integer from 1 to 3, 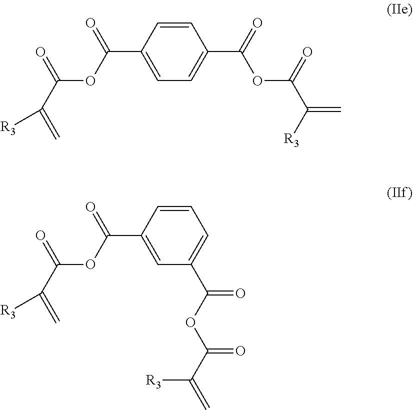
where R₃ is independently H or methyl.

7. The method according to claim 1, wherein the temporary crosslinker is methacrylic anhydride or acrylic anhydride.

8. The method according to claim 1, wherein the permanent crosslinker has two or more vinyl groups.

9. The method according to claim 1, wherein the permanent crosslinker is selected from the group consisting of divinylbenzene (DVB), ethylene glycol dimethacrylate (EGDMA), diethyleneglycol dimethacrylate (DEGDMA), N,N′-methylenebisacrylamide (MBA), oligo/poly ethyleneglycol dimethyacrylate, 1,4-butanediol dimethacrylate, and 1,6-hexanediol dimethacrylate.

10. The method according to claim 1, wherein the permanent crosslinker is between 1 to 30 mol % of the total monomer loading.

11. (canceled)

12. The method according to claim 1, wherein the step of combining comprises combining a photoinitiator and the method further comprises irradiating the photoinitiator.

13. The method according to claim 1, wherein the precipitation polymerization is performed without the addition of surfactant and/or stabilizer, and/or the microparticles comprise less than 1% surfactant and/or stabilizer.

14. The method according to claim 1, further comprising functionalizing the monomers of the temporary crosslinkers.

15. The method according to claim 14, wherein the step of functionalizing comprises functionalizing to obtain amines and carboxylic acid units in a ratio of 3:1 to 1:3.

16. The method according to claim 15, wherein the ratio is between 2:1 to 1:2.

17. Microparticles comprising at least one polymer, the at least one polymer comprising:
temporary crosslinker units as defined in formula (Illa), (Illb), (Illc), (Illd), (Ille), (Illf), (Illg), (Illh), (Illi), (Illj), (Illk), (Illl), (Illm), (Illn), and/or (Illo): 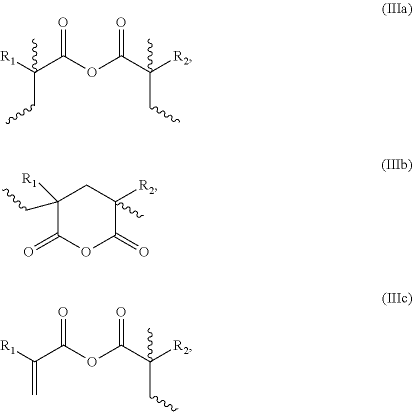
wherein R₁ and R₂ are are independently selected from H, C₁-C₄ linear or branched carbon chain, benzyl, phenyl or OJ, and wherein J is defined as a C₁-C₄ linear or branched carbon chain, 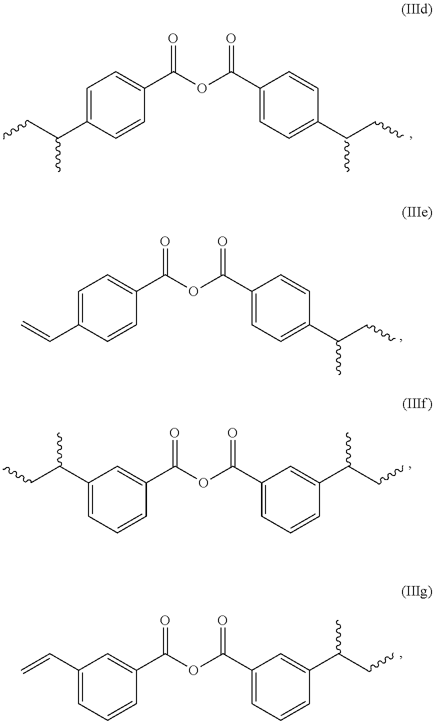 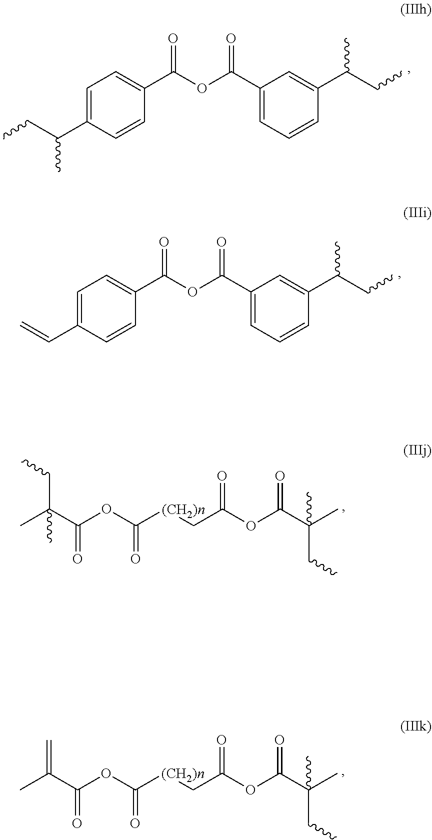
wherein n is an integer from 1 to 3, 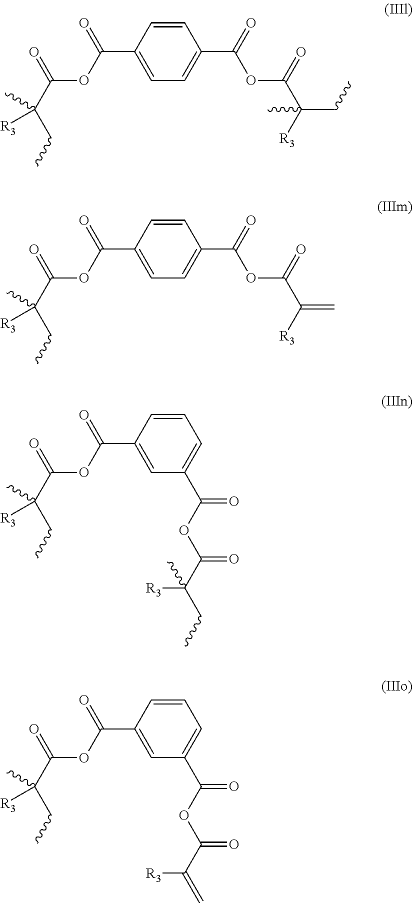
where R₃ is independently H or methyl;
permanent crosslinker units;
wherein the microparticles are narrow-disperse or mono-disperse and have a size distribution having a coefficient of variation of less than 0.3.

18-27. (canceled)

28. A method of cryopreserving cells comprising:
providing microparticles obtained by the method as defined in claim 1;
functionalizing the microparticles;
contacting the cells with the microparticles; and
freezing the cells.

29. A method of producing a vaccine delivery platform comprising:
providing microparticles obtained by the method as defined in claim 1;
functionalizing the microparticles to act as a carrier for an antigen; and
associating the antigen to the carrier.

30. A method of producing encapsulated cells comprising:
providing the microparticles obtained by the method as defined in claim 1;
functionalizing the microparticles;
combining functionalized microparticles with cells and a capsule-forming material; and
gelling the capsule-forming material such that the particles and cells become entrapped within the capsule.

31. The method according to claim 30, wherein the capsule-forming material is alginate.

32-44. (canceled)
